(19) United States
(12) Patent Application Publication
(10) Pub. No.: US 2024/0350666 A1
(43) Pub. Date: Oct. 24, 2024

(54) COMPOSITIONS AND METHODS FOR HIGH EFFICIENCY GENOME EDITING

(71) Applicant: Children`s Medical Center Corporation, Boston, MA (US)

(72) Inventors: William Pu, Chestnut Hill, MA (US); Nathan VanDusen, Brookline, MA (US); Suya Wang, Boston, MA (US); Mason Eric Sweat, Brookline, MA (US)

(21) Appl. No.: 18/685,599

(22) PCT Filed: Aug. 23, 2022
(86) PCT No.: PCT/US22/41223

§ 371 Date: Feb. 22, 2024

Related U.S. Application Data
(60) Provisional application No. 63/235,989, filed on Aug. 23, 2021.

(51) Int. Cl. A61K 48/00; A61P 9/00; A61P 21/00; C12N 9/22; C12N 15/11; C12N 15/86

(52) U.S. Cl. CPC A61K 48/005; A61P 9/00; A61P 21/00; C12N 9/22; C12N 15/111; C12N 15/86; C12N 2310/20; C12N 2740/15043; C12N 2750/14143

(57) ABSTRACT

Provided herein are compositions and methods for high efficiency genome editing by targeting novel genetic loci.

### CLAIM OF PRIORITY

This application claims the benefit of U.S. Provisional Application Ser. No. 63/235,989, filed on Aug. 23, 2021, the entire content of which is incorporated herein by reference.

### FEDERALLY SPONSORED RESEARCH OR DEVELOPMENT

This invention was made with government support under grant numbers K99HL143194 and R01HL146634 awarded by the National Institutes of Health (NIH), and 2UM1HL098166 awarded by the National Heart, Lung, and Blood Institute. The government has certain rights in the invention.

### TECHNICAL FIELD

This disclosure relates to novel loci and methods for highly efficient, precise, in vivo somatic genome modification.

### BACKGROUND

Recent advances in genome sequencing techniques and analysis methods have significantly accelerated the ability to catalog and map genetic factors associated with a diverse range of biological functions and diseases. Precise genome targeting technologies are needed to enable systematic reverse engineering of causal genetic variations by allowing selective perturbation of individual genetic elements, as well as to advance synthetic biology, biotechnological, and medical applications.

CRISPR/Cas9-based genome editing technologies provide powerful tools for genetic manipulation. Delivery of Cas9 and a homology directed repair (HDR) template using adeno-associated virus (AAV; CASAAV-HDR) was recently shown to enable creation of precise genomic edits, even within postmitotic cells.

Therefore, there is a need in the art to identify novel loci that allow high efficiency genome editing.

### SUMMARY

Provided herein are compositions and methods for high efficiency genome editing by homology directed repair targeting newly identified loci.

Accordingly, provided herein is a method for integrating an exogenous sequence into a chromosomal sequence of a eukaryotic cell, the method comprising: a. introducing into the eukaryotic cell: (i) at least one RNA-guided endonuclease comprising at least one nuclear localization signal or nucleic acid encoding at least one RNA-guided endonuclease comprising at least one nuclear localization signal, (ii) at least one guide RNA or a DNA encoding at least one guide RNA, and (iii) at least one donor polynucleotide comprising the exogenous sequence; b. generating a double-stranded break at a target site in the chromosomal sequence, wherein at least one guide RNA guides RNA-guided endonuclease to the target site; and c. repairing the double strand break using a DNA repair process, thereby integrating the exogenous sequence into the chromosomal sequence of the eukaryotic cell, wherein the efficiency of integrating the exogenous sequence is about 20%, 25%, 30%, 35%, 40%, or 45% higher compared to a reference sample.

In some embodiments, the eukaryotic cell is a cardiomyocyte or a skeletal muscle cell.

In some embodiments, the insertion site for the exogenous sequence is selected from the group consisting of: Myl2, Myl7, Pln, Ttn.

In some embodiments, the exogenous sequence is integrated into the 5′ or 3′ of Myl2.

In some embodiments, the exogenous sequence is integrated into the 5′ or 3′ of Pln.

In some embodiments, the insertion site for the exogenous sequence is selected from the group consisting of: Mb, Des, Actc1, Cox6a2, Fabp3, Myh6, Rplp1, Acta1, Myl3, Myl2, Myl7, Pln, and Ttn.

In some embodiments, the exogenous sequence is integrated into the 5′ or 3′ of Mb.

In some embodiments, the exogenous sequence is integrated into the 5′ or 3′ of Des.

In one aspect, provided herein is a homology directed repair (HDR) construct comprising a left and right homology arm for a genomic edit to be incorporated at a target locus.

In some embodiments, the target locus is selected from the group consisting of: Myl2, Myl7, Pln, and Ttn.

In some embodiments, the genomic edit is incorporated into the 5′ or 3′ of Myl2.

In some embodiments, the genomic edit is incorporated into the 5′ or 3′ of Pln.

In some embodiments, the target locus is selected from the group consisting of: Mb, Des, Actc1, Cox6a2, Fabp3, Myh6, Rplp1, Acta1, Myl3, Myl2, Myl7, Pln, and Ttn.

In some embodiments, the genomic edit is incorporated into the 5′ or 3′ of Mb.

In some embodiments, the genomic edit is incorporated into the 5′ or 3′ of Des.

In some embodiments, the HDR construct further comprises a positive selection or negative selection marker.

In some embodiments, the HDR construct comprises a fluorescent marker for FACS isolation of positive cell pools, wherein the fluorescent marker comprises mScarlet, Blue-TagBFP, Cyan-Cerulean, Green-Tag GFP2, Yellow-YPet, Red-TagRFP, Far Red-mKate2.

In one aspect, provided herein is a homology directed repair (HDR) vector comprising any of the construct described herein.

In some embodiments, the backbone of the vector enables uniform, one-step assembly for incorporating homology arms.

In some embodiments, the vector is a transfection delivery vector.

In some embodiments, the vector is a viral delivery vector.

In some embodiments, the viral delivery vector is a lentivirus vector.

In some embodiments, the viral vector is an AAV vector.

In some embodiments, the AAV vector is an AAV9 vector.

In one aspect, provided herein is an engineered, non-naturally occurring CRISPR-Cas system comprising: a Cas9 protein which is a Streptococcus pyogenes Cas9 comprising mutation or an ortholog thereof having a corresponding mutation, and an HDR vector described herein.

In one aspect, provided herein is an isolated, engineered, non-naturally occurring cell comprising a CRISPR-Cas system described herein.

In some embodiments, the cell is a eukaryotic cell.

In some embodiments, the cell is a mammalian cell.

In some embodiments, the cell is a cardiomyocyte.

In some embodiments, the cell is a skeleton muscle cell.

In one aspect, provided herein is a method of treating a disease in a subject, comprising administering an effective amount of the HDR construct described herein, an HDR vector described herein, or the engineered non-naturally occurring CRISPR-Cas system described herein to the subject, thereby treating the subject.

In some embodiments, the subject is a human subject.

In some embodiments, the disease is a cardiomyopathy.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting.

All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a schematic illustration of CASAAV-mediated gene editing via non-homologous end-joining of the double strand break introduced by CRISPR.

FIG. 2A depicts HDR-mediated repair of a double strand break introduced by CRISPR, resulting in locus-specific transgene integration.

FIG. 2B shows that CASAAV-HDR vectors targeting Myl2 or Myl7 resulted in mScarlet expression specifically in ventricular (V) or atrial (A) heart chambers. Scale bar=50 μm.

FIG. 2C shows the CASAAV-HDR dose response. AAV was administered at high, mid, and low doses (5×10¹¹, 5×10¹⁰, and 5×10⁹ vg/g respectively), resulting in approximately 96%, 83%, and 51% myocardial transduction respectively. Transduced cardiomyocytes, dissociated by Langendorff perfusion, were analyzed for GFP (transduction marker) and RFP (HDR marker) expression by flow cytometry. *: p-value<0.001; **: p-value<0.01.

FIG. 2D shows that CASAAV-HDR integration of P2A-mScarlet at Myl2 depends on Cas9 and homology arms. i. AAV9-HDR-Myl2: CASAAV-HDR vector targeting Myl2 was delivered to Tnnt2Cre; Rosa26fsCas9 mice. ii. no Cas9: vector was delivered to mice lacking Cas9 expression. iii. No homol. arms: AAV similar to AAV9-HDR-Myl2 but lacking homology arms was delivered to Tnnt2Cre; Rosa26fsCas9 mice. iv. AAV-Cas9, AAV9-HDR-Myl2 plus AAV9-Tnnt2-Cas9 were delivered to wild type mice. Scale bar=50 μm.

FIGS. 2E-2F show the results of co-injection of AAVHDR-Myl2-mScarlet and AAV-Cas9 showing RFP expression selectively in ventricles, indicative of HDR at the Myl2 locus.

FIG. 3A shows that Cas9-induced double strand breaks can be repaired by homology-directed repair (HDR) or nonhomologous end joining (NHEJ). Blue lines, homology arms; open arrows, primers used for amplicon sequencing. Unless noted otherwise, AAV9 was delivered subcutaneously at P0 and hearts were analyzed at P7.

FIG. 3B shows the quantification of mutations induced by high dose AAV and Cas9-mediated HDR insertion of P2A-mScarlet into the C-terminus of Myl2 or Myl7. The junctions between inserted sequence and endogenous sequence were amplified from cDNA. Primers are illustrated in (A). For alleles lacking an insert, a fragment was amplified from DNA using primers flanking the gRNA target site. Amplicons were deeply sequenced and analyzed for the indicated types of modifications.

FIG. 4A is a schematic illustration of the experimental design of Myl2 HDR efficiency in fetal, neonatal, or mature cardiomyocytes.

FIG. 4B shows Myl2 HDR efficiency in fetal, neonatal, or mature cardiomyocytes. AAV was administered at equivalent “Mid” dose (5×10¹0 vg/g; E15.5 embryo=0.6 g) at each stage, resulting in 80-83% myocardial transduction. mScarlet-expressing cardiomyocytes were quantified by flow cytometry. Differences between groups were not significant. CMs: cardiomyocytes.

FIGS. 4C-4D show efficient fetal cardiac transduction by AAV9 vectors. AAV-Cre recombined the Rosa26mTmG allele, switching expression from RFP to GFP.

FIGS. 5A-5B show that AAV-HDR enables analysis of protein localization. YAP1 and Mkl2 endogenous genes were epitope tagged with HA and HA visualized by immunostaining. PLN and TTN endogenous genes were labeled with mScarlet and red fluorescence was detected in dissociated adult cardiomyocytes.

FIG. 6 shows the summary of HDR efficiency at 9 different loci, as a function of gene expression level in P0 ventricular cardiomyocytes, or atrial cardiomyocytes in the case of Myl7. Homology arms were approximately 1 kb long. Shading indicates 95% confidence interval for fitted line.

FIGS. 7A-7B show that AAVHDR efficiency primarily occurs at loci actively expressing the targeted gene. Myl2 is expressed in ventricle but not liver. Genomic DNA analysis by PCR shows that HDR was more efficient in ventricle than liver, whereas viral transduction was robust in both tissues.

FIG. 8 is a panel of microscopic images showing the results of co-injection of AAV-CAG-Luciferase (luciferase) and AAV-HDR-Des-mScarlet at P3 (mediating Desmin stop codon replacement by P2A-mScarlet), examined at 18 days or 3 months after injection. Episomal luciferase expression declined in skeletal muscle but genomic Desmin-P2A-mScarlet expression stably persisted.

FIG. 9 is a schematic illustration of a vector for use in the treatment of CPVT by therapeutic integration of AIP at Myl2 3′ UTR. SNAP is a reporter gene that is readily imaged.

FIGS. 10A-10C show the AAVHDR efficiency in cardiomyocytes by targeting the Myl2 locus with P2A-SNAP or P2A-SNAP-AIP.

FIG. 11 shows the production of SNAP proteins after AAVHDR targeting of P2A-SNAP or P2A-SNAP-AIP to the Myl2 locus. Cardiac lysates were analyzed by capillary western. An Myl2-SNAP fusion protein with the expected size of ˜40 kDa was not observed indicating efficient separation of Myl2 and SNAP proteins by P2A.

FIGS. 12A-12B show that HDR-SNAP-AIP at the Myl2 locus reduces arrhythmia burden in CPVT (Ryr2^{R4650I/}) mice. NSVT, non-sustained VT (>=3 ventricular beats in a row).

FIGS. 13A-13B show that HDR-SNAP-AIP at the Myl2 locus rescues abnormal calcium handling of isolated CPVT (Ryr2^{R4650I/}) cardiomyocytes. Cardiomyocyte cytosolic Ca2+ was imaged using X-Rhod. Cells were paced at 1 Hz and then pacing was abruptly stopped. Normal cardiomyocytes have rare Ca2+ waves after pacing, CPVT cardiomyocytes have spontaneous Ca2+ waves, and SNAP-AIP expression from the Myl2 locus suppresses the spontaneous Ca2+ waves.

FIG. 14 shows the effect of HDR at the Myl2 locus on systolic ventricular function. Myl2 modification resulted in systolic dysfunction (reduced FS %) and ventricular dilatation.

FIG. 15 is a schematic illustration of an example HDR vector described herein.

FIGS. 16A-16B show the HDR efficiency for loci Actc1, Cox6a2, Fabp3, Mb, Myh6, and Rplp1.

FIG. 17 shows heart function after HDR modification of the indicated loci with respective the AAVHDR vectors. Heart function was measured by echocardiography.

FIG. 18 shows the HDR efficiency in heart by targeting Des, Acta1, Myl2 and Myl3. HT, heart. SK, skeletal muscle.

FIG. 19 shows the HDR efficiency for Des and Mb loci in heart (HT) and skeletal muscle (SK).

FIG. 20 shows that Desmin HDR occurred in both slow and fast fibers. Mb HDR was highly efficient in slow fibers and also occurred in some fast fibers.

FIGS. 21A-21C show that Desmin HDR did not cause cardiac toxicity. Desmin targeting in heart does not affect ventricular size or function.

FIG. 22 shows the therapeutic efficacy of HDR-mediated editing for Barth syndrome, caused by lack of the gene TAZ. AAVHDR was used to target the desmin locus with P2A-TAZ to restore cardiac TAZ expression. Heart function was measured by echocardiography. * or #, P<0.05. ###, P<0.001. ****, P<0.0001.

FIGS. 23A-23D show that at higher doses MyoAAV has equivalent HDR efficiency at Mb to AAV9 for heart. But MyoAAV2A is better for skeletal muscle.

FIG. 24 shows that at lower doses, MyoAAV2A was superior to AAV9 in both heart and skeletal muscle, for both transduction (GFP) and HDR at Mb (Halo).

FIG. 25 shows that mice treated with 1×10¹¹ VG/G packaged with either capsid and mediating HDR at Mb had good heart function at P31.

### DETAILED DESCRIPTION

Disclosed here are novel loci for high efficiency gene editing. The efficiency of in vivo CRISPR/AAV-mediated HDR is especially high in cardiomyocytes. These novel loci, e.g., Mb, Des, Actc1, Cox6a2, Fabp3, Myh6, Rplp1, Acta1, Myl3, Myl2, Myl7, Pln, and Ttn, allow precise gene editing, e.g., exogenous gene insertion into the target sites. These loci also allow effective gene editing in both proliferating and non-proliferating cells, e.g., cardiomyocytes and skeletal muscle cells. In addition, the vectors targeting these loci are useful for monitoring protein localization. The novel loci described herein, e.g., Mb and Des are also promising loci for therapeutic transgene expression.

The novel HDR-based gene editing systems described herein are also particularly useful for skeletal muscle diseases (e.g. Duchenne), since viral dilution is more problematic for skeletal muscle disease.

The novel HDR-based gene editing systems described herein are also useful for applications such as fetal or neonatal AAV gene therapy, which have been challenging due to problems caused by viral dilution.

As used herein, “CASAAVHDR,” “CASAAV-HDR,” or “CAS/AAV/HDR” refers to adenovirus (AAV)-mediated, homology-directed repair (HDR)-based, CRISPR systems. The DNA repair process is described, e.g., in The Cell: A Molecular Approach. 2nd edition, the entire content of which is incorporated by reference herein.

### Loci for Integration

### Myl2 and Myl7

Myosins are a large family of motor proteins that share the common features of ATP hydrolysis (ATPase enzyme activity), actin binding and potential for kinetic energy transduction. Originally isolated from muscle cells, almost all eukaryotic cells are known to contain myosins. Following phosphorylation, it plays a role in cross-bridge cycling kinetics and cardiac muscle contraction by increasing myosin lever arm stiffness and promoting myosin head diffusion; as a consequence of the increase in maximum contraction force and calcium sensitivity of contraction force. These events altogether slow down myosin kinetics and prolong duty cycle resulting in accumulated myosins being cooperatively recruited to actin binding sites to sustain thin filament activation as a means to fine-tune myofilament calcium sensitivity to force. During cardiogenesis plays an early role in cardiac contractility by promoting cardiac myofibril assembly.

Myl2 (NCBI Reference Sequence: NG_007554.1) is a protein coding gene encoding myosin light chain 2. Myl7 (NCBI Reference Sequence: NM_021223.3) encodes myosin regulatory light chain 2 and myosin, light polypeptide 7. Myosin is a contractile protein that plays a role in heart development and function. Thus gene encodes the regulatory light chain associated with cardiac myosin beta (or slow) heavy chain. Ca+ triggers the phosphorylation of regulatory light chain that in turn triggers contraction. Mutations in this gene are associated with mid-left ventricular chamber type hypertrophic cardiomyopathy.

Diseases associated with Myl2 and Myl7 include cardiomyopathy, familial hypertrophic, and congenital fiber-type disproportion. Among its related pathways are RhoGDI Pathway and PAK Pathway. Gene Ontology (GO) annotations related to this gene include calcium ion binding and actin monomer binding. An important paralog of this gene is myl10.

In some embodiments, the integration sites for exogenous gene in the methods described herein is Myl2. In some embodiments, the integration sites for exogenous gene in the methods described herein is Myl7.

### Pln

The Pln gene (NCBI Reference Sequence: NM_002667.5) encodes cardiac phospholamban. It reversibly inhibits the activity of ATP2A2 in cardiac sarcoplasmic reticulum by decreasing the apparent affinity of the ATPase for Ca²⁺ (PubMed: 28890335).

Cardiac phospholamban modulates the contractility of the heart muscle in response to physiological stimuli via its effects on ATP2A2. Modulates calcium re-uptake during muscle relaxation and plays an important role in calcium homeostasis in the heart muscle. The degree of ATP2A2 inhibition depends on the oligomeric state of PLN. ATP2A2 inhibition is alleviated by PLN phosphorylation.

In some embodiments, the integration sites for exogenous gene in the methods described herein is Pln.

### Ttn

The Ttn gene (NCBI Reference Sequence: NM_001267550.2) encodes a very large protein called Titin. This protein plays an important role in muscles the body uses for movement (skeletal muscles) and in heart (cardiac) muscle. Slightly different versions (called isoforms) of Titin are made in different muscles.

Within muscle cells, Titin is an essential component of structures called sarcomeres. Sarcomeres are the basic units of muscle contraction; they are made of proteins that generate the mechanical force needed for muscles to contract. Titin has several functions within sarcomeres. One of the protein's main jobs is to provide structure, flexibility, and stability to these cell structures. Titin interacts with other muscle proteins, including actin and myosin, to keep the components of sarcomeres in place as muscles contract and relax. Titin also contains a spring-like region that allows muscles to stretch. Additionally, researchers have found that titin plays a role in chemical signaling and in assembling new sarcomeres.

In some embodiments, the integration sites for exogenous gene in the methods described herein is Ttn.

### Mb

Myoglobin is a protein that's found in the striated muscles, which includes skeletal muscles and heart muscles. Its main function is to supply oxygen to the cells in the muscles (myocytes).

Mb (NCBI Reference Sequence: NG_007075.1) encodes a member of the globin superfamily and is predominantly expressed in skeletal and cardiac muscles. The encoded protein forms a monomeric globular haemoprotein that is primarily responsible for the storage and facilitated transfer of oxygen from the cell membrane to the mitochondria. This protein also plays a role in regulating physiological levels of nitric oxide. Multiple transcript variants encoding distinct isoforms exist for this gene.

In some embodiments, the integration sites for exogenous gene in the methods described herein is Mb.

### Des

Des (NCBI Reference Sequence: NG_008043.1) encodes desmin, a muscle-specific class III intermediate filament. Homopolymers of this protein form a stable intracytoplasmic filamentous network connecting myofibrils to each other and to the plasma membrane. Mutations in this gene are associated with desmin-related myopathy, a familial cardiac and skeletal myopathy (CSM), and with distal myopathies.

In some embodiments, the integration sites for exogenous gene in the methods described herein is Des.

### Actc1

Actc1 (NCBI Reference Sequence: NG_007553.1) encodes actin alpha cardiac muscle 1. Actins are highly conserved proteins that are involved in various types of cell motility. Polymerization of globular actin (G-actin) leads to a structural filament (F-actin) in the form of a two-stranded helix. Each actin can bind to four others. The protein encoded by this gene belongs to the actin family which is comprised of three main groups of actin isoforms, alpha, beta, and gamma. The alpha actins are found in muscle tissues and are a major constituent of the contractile apparatus. Defects in this gene have been associated with idiopathic dilated cardiomyopathy (IDC) and familial hypertrophic cardiomyopathy (FHC).

In some embodiments, the integration sites for exogenous gene in the methods described herein is Actc1.

### Cox6a2

Cox6a2 (NCBI Reference Sequence: NC_000016.10) encodes cytochrome c oxidase subunit 6A2. Cytochrome c oxidase (COX), the terminal enzyme of the mitochondrial respiratory chain, catalyzes the electron transfer from reduced cytochrome c to oxygen. It is a heteromeric complex consisting of 3 catalytic subunits encoded by mitochondrial genes and multiple structural subunits encoded by nuclear genes. The mitochondrially-encoded subunits function in electron transfer, and the nuclear-encoded subunits may be involved in the regulation and assembly of the complex. This nuclear gene encodes polypeptide 2 (heart/muscle isoform) of subunit VIa, and polypeptide 2 is present only in striated muscles. Polypeptide 1 (liver isoform) of subunit VIa is encoded by a different gene, and is found in all non-muscle tissues. These two polypeptides share 66% amino acid sequence identity.

In some embodiments, the integration sites for exogenous gene in the methods described herein is Cox6a2.

### Fabp3

Fabp3 (NCBI Reference Sequence: NG_047049.1) encodes fatty acid binding protein 3. The intracellular fatty acid-binding proteins (FABPs) belongs to a multigene family. FABPs are divided into at least three distinct types, namely the hepatic-, intestinal- and cardiac-type. They form 14-15 kDa proteins and are thought to participate in the uptake, intracellular metabolism and/or transport of long-chain fatty acids. They may also be responsible in the modulation of cell growth and proliferation. Fatty acid-binding protein 3 gene contains four exons and its function is to arrest growth of mammary epithelial cells. This gene is a candidate tumor suppressor gene for human breast cancer. Alternative splicing results in multiple transcript variants.

In some embodiments, the integration sites for exogenous gene in the methods described herein is Fabp3.

### Myh6

Myh6 (NCBI Reference Sequence: NG_023444.1) encodes myosin heavy chain 6. Cardiac muscle myosin is a hexamer consisting of two heavy chain subunits, two light chain subunits, and two regulatory subunits. This gene encodes the alpha heavy chain subunit of cardiac myosin. The gene is located approximately 4 kb downstream of the gene encoding the beta heavy chain subunit of cardiac myosin. Mutations in this gene cause familial hypertrophic cardiomyopathy and atrial septal defect 3.

In some embodiments, the integration sites for exogenous gene in the methods described herein is Myh6.

### Rplp1

Rplp1 (NCBI Reference Sequence: NC_000015.10) encodes ribosomal protein lateral stalk subunit P1.

Ribosomes, the organelles that catalyze protein synthesis, consist of a small 40S subunit and a large 60S subunit. Together these subunits are composed of 4 RNA species and approximately 80 structurally distinct proteins. This gene encodes a ribosomal phosphoprotein that is a component of the 60S subunit. The protein, which is a functional equivalent of the E. coli L7/L12 ribosomal protein, belongs to the L12P family of ribosomal proteins. It plays an important role in the elongation step of protein synthesis. Unlike most ribosomal proteins, which are basic, the encoded protein is acidic. Its C-terminal end is nearly identical to the C-terminal ends of the ribosomal phosphoproteins P0 and P2. The P1 protein can interact with P0 and P2 to form a pentameric complex consisting of P1 and P2 dimers, and a P0 monomer. The protein is located in the cytoplasm. Two alternatively spliced transcript variants that encode different proteins have been observed. As is typical for genes encoding ribosomal proteins, there are multiple processed pseudogenes of this gene dispersed through the genome.

In some embodiments, the integration sites for exogenous gene in the methods described herein is Rplp1.

### Acta1

Acta1 (NCBI Reference Sequence: NG_006672.1) encodes actin alpha 1, skeletal muscle. The product encoded by this gene belongs to the actin family of proteins, which are highly conserved proteins that play a role in cell motility, structure and integrity. Alpha, beta and gamma actin isoforms have been identified, with alpha actins being a major constituent of the contractile apparatus, while beta and gamma actins are involved in the regulation of cell motility. This actin is an alpha actin that is found in skeletal muscle. Mutations in this gene cause a variety of myopathies, including nemaline myopathy, congenital myopathy with excess of thin myofilaments, congenital myopathy with cores, and congenital myopathy with fiber-type disproportion, diseases that lead to muscle fiber defects with manifestations such as hypotonia.

In some embodiments, the integration sites for exogenous gene in the methods described herein is Acta1.

### Myl3

Myl3 (NCBI Reference Sequence: NG_007555.2) encodes myosin light chain 3, an alkali light chain also referred to in the literature as both the ventricular isoform and the slow skeletal muscle isoform. Mutations in MYL3 have been identified as a cause of mid-left ventricular chamber type hypertrophic cardiomyopathy.

In some embodiments, the integration sites for exogenous gene in the methods described herein is Myl3.

### Homology-Directed Repair (HDR) and CRISPR/Cas9 Systems for Genome Editing

Recent advances in genome sequencing techniques and analysis methods have significantly accelerated the ability to catalog and map genetic factors associated with a diverse range of biological functions and diseases. Precise genome targeting technologies are needed to enable systematic reverse engineering of causal genetic variations by allowing selective perturbation of individual genetic elements, as well as to advance synthetic biology, biotechnological, and medical applications. Although genome-editing techniques such as designer zinc fingers, transcription activator-like effectors (TALEs), or homing meganucleases are available for producing targeted genome perturbations, there remains a need for new genome engineering technologies that are affordable, easy to set up, scalable, and amenable to targeting multiple positions within the eukaryotic genome.

Targeted, rapid, and efficient genome editing using the RNA-guided Cas9 system is enabling the systematic interrogation of genetic elements in a variety of cells and organisms and holds enormous potential as next-generation gene therapies. In contrast to other DNA targeting systems based on zinc-finger proteins (ZFPs) and transcription activator-like effectors (TALEs), which rely on protein domains to confer DNA-binding specificity, Cas9 forms a complex with a small guide RNA that directs the enzyme to its DNA target via Watson-Crick base pairing. Consequently, the system is simple and fast to design and requires only the production of a short oligonucleotide to direct DNA binding to any locus.

The type II microbial CRISPR (clustered regularly interspaced short palindromic repeats) system, which is the simplest among the three known CRISPR types, consists of the CRISPR-associated (Cas) genes and a series of non-coding repetitive elements (direct repeats) interspaced by short variable sequences (spacers). These short approximate 30 bp spacers are often derived from foreign genetic elements such as phages and conjugating plasmids, and they constitute the basis for an adaptive immune memory of those invading elements. The corresponding sequences on the phage genomes and plasmids are called protospacers, and each protospacer is flanked by a short protospacer-adjacent motif (PAM), which plays a critical role in the target search and recognition mechanism of Cas9. The CRISPR array is transcribed and processed into short RNA molecules known as CRISPR RNAs (crRNA) that, together with a second short trans-activating RNA (tracrRNA), complex with Cas9 to facilitate target recognition and cleavage. Additionally, the crRNA and tracrRNA can be fused into a single guide RNA (sgRNA) to facilitate Cas9 targeting.

The Cas9 enzyme from Streptococcus pyogenes (SpCas9), which requires a 5′-NGG PAM, has been widely used for genome editing applications (Hsu et al., 2014). In order to target any desired genomic locus of interest that fulfills the PAM requirement, the enzyme can be “programmed” merely by altering the 20-bp guide sequence of the sgRNA. Additionally, the simplicity of targeting lends itself to easy multiplexing such as simultaneous editing of several loci by including multiple sgRNAs.

Like other designer nucleases, Cas9 facilitates genome editing by inducing double-strand breaks (DSBs) at its target site, which in turn stimulates endogenous DNA damage repair pathways that lead to edited DNA: homology directed repair (HDR), which requires a homologous template for recombination but repairs DSBs with high fidelity, and non-homologous end-joining (NHEJ), which functions without a template and frequently produces insertions or deletions (indels) as a consequence of repair. Exogenous HDR templates can be designed and introduced along with Cas9 and sgRNA to promote exact sequence alteration at a target locus; however, this process is conventionally held to occur only in dividing cells and at low efficiency.

Certain applications—e.g., therapeutic genome editing in human stem cells—demands editing that is not only efficient, but also highly specific. Nucleases with off-target DSB activity could induce undesirable mutations with potentially deleterious effects, an unacceptable outcome in most clinical settings. The remarkable ease of targeting Cas9 has enabled extensive off-target binding and mutagenesis studies employing deep sequencing and chromatin immunoprecipitation (ChIP) in human cells. As a result, an increasingly complete picture of the off-target activity of the enzyme is emerging. Cas9 will tolerate some mismatches between its guide and a DNA substrate, a characteristic that depends strongly on the number, position (PAM proximal or distal) and identity of the mismatches. Off-target binding and cleavage may further depend on the organism being edited, the cell type, and epigenetic contexts.

These specificity studies, together with direct investigations of the catalytic mechanism of Cas9, have stimulated homology- and structure-guided engineering to improve its targeting specificity. The wild-type enzyme makes use of two conserved nuclease domains, HNH and RuvC, to cleave DNA by nicking the sgRNA-complimentary and non-complimentary strands, respectively. A “nickase” mutant (Cas9n) can be generated by alanine substitution at key catalytic residues within these domains—SpCas9 D10A inactivates RuvC, while N863A has been found to inactivate HNH. Though an H840A mutation was also reported to convert Cas9 into a nicking enzyme, this mutant has reduced levels of activity in mammalian cells compared with N863A.

Because single stranded nicks are generally repaired via the non-mutagenic base-excision repair pathway, Cas9n mutants can be leveraged to mediate highly specific genome engineering. A single Cas9n-induced nick can stimulate HDR at low efficiency in some cell types, while two nicking enzymes, appropriately spaced and oriented at the same locus, effectively generate DSBs, creating 3′ or 5′ overhangs along the target as opposed to a blunt DSB as in the wild-type case. The on-target modification efficiency of the double-nicking strategy is comparable to wild-type, but indels at predicted off-target sites are reduced below the threshold of detection by Illumina deep sequencing.

Despite this progress in Cas9 directed genetic engineering technologies, the efficiency of successful gene modifications, in particular in the context of HDR, is still at low levels, and improved strategies for increasing HDR efficiency for Cas9 directed genetic engineering are needed.

Detailed description of suitable CRIPR-Cas9 systems that can be used in the systems and methods described herein are disclosed, e.g., in US20200354751A1, the entire content of which is incorporated by reference herein.

Described herein are CRISPR-Cas9 systems that include an HDR vector for precise targeting of a genetic locus. In some embodiments, the genetic locus is Myl2. In some embodiments, the genetic locus is Des. In some embodiments, the genetic locus is Pln. In some embodiments, the genetic locus is Mb.

In some embodiments and as discussed below, the vector is a viral vector. In some embodiments, the vector is a lentiviral vector. In some embodiments, the vector is an AAV vector.

### Adeno-Associated Virus (AAV)

Adeno-associated virus (AAV) has shown promise for delivering genes for gene therapy in clinical trials in humans. As the only viral vector system based on a nonpathogenic and replication-defective virus, recombinant AAV virions have been successfully used to establish efficient and sustained gene transfer of both proliferating and terminally differentiated cells in a variety of tissues.

The AAV genome is a linear, single-stranded DNA molecule containing about 4681 nucleotides. The AAV genome generally comprises an internal nonrepeating genome flanked on each end by inverted terminal repeats (ITRs). The ITRs are approximately 145 base pairs (bp) in length. The ITRs have multiple functions, including as origins of DNA replication, and as packaging signals for the viral genome. The internal nonrepeated portion of the genome includes two large open reading frames, known as the AAV replication (rep) and capsid (cap) genes. The rep and cap genes code for viral proteins that allow the virus to replicate and package into a virion. In particular, a family of at least four viral proteins is expressed from the AAV rep region, Rep 78, Rep 68, Rep 52, and Rep 40, named according to their apparent molecular weight. The AAV cap region encodes at least three proteins, VP1, VP2, and VP3.

AAV has been engineered to deliver genes of interest by deleting the internal nonrepeating portion of the AAV genome (i.e., the rep and cap genes) and inserting a heterologous gene between the ITRs. The heterologous gene is typically functionally or operatively linked to a heterologous promoter (constitutive, cell-specific, or inducible) capable of driving gene expression in the patient's target cells under appropriate conditions. Termination signals, such as polyadenylation sites, can also be included.

As used herein, the term “AAV vector” means a vector derived from an adeno-associated virus serotype, including without limitation, AAV1, AAV2, AAV3, AAV4, AAV5, AAV6, AAV7, AAV8, AAV9, and mutated forms thereof. In some instances, AAV9 is used. AAV vectors can have one or more of the AAV wild-type genes deleted in whole or part, the rep and/or cap genes, but retain functional flanking ITR sequences. In some embodiments, the AAV vector is derived from an adeno-associated virus serotype AAV1. Despite the high degree of homology, the different serotypes have tropisms for different tissues. The receptor for AAV1 is unknown; however, AAV1 is known to transduce skeletal and smooth muscle more efficiently than AAV2. Without being bound by theory, since most of the studies have been done with pseudotyped vectors in which the vector DNA flanked with AAV2 ITR is packaged into capsids of alternate serotypes, it is clear that the biological differences are related to the capsid rather than to the genomes. Recent evidence indicates that DNA expression cassettes packaged in AAV1 capsids are at least 1 log10 more efficient at transducing cardiomyocytes than those packaged in AAV2 capsids.

Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, for example, by the insertion, deletion or substitution of nucleotides, as long as the sequences provide for functional rescue, replication and packaging.

The ITR consists of nucleotides 1 to 145 at the left end of the AAV DNA genome and the corresponding nucleotides 4681 to 4536 (i.e., the same sequence) at the right hand end of the AAV DNA genome. Thus, AAV vectors must have a total of at least 300 nucleotides of the terminal sequence. So, for packaging large coding regions into AAV vector particles, it is important to develop the smallest possible regulatory sequences, such as transcription promoters and poly A addition signal. In this system, the adeno-associated viral vector comprising the inverted terminal repeat (ITR) sequences of adeno-associated virus and a nucleic acid encoding Myl2, mly7, or one of its isoforms, fragments and/or variants, wherein the inverted terminal repeat sequences promote expression of the nucleic acid in the absence of another promoter.

Accordingly, as used herein, AAV refers to all serotypes of AAV (i.e., 1-9) and mutated forms thereof. Thus, it is routine in the art to use the ITR sequences from other serotypes of AAV since the ITRs of all AAV serotypes are expected to have similar structures and functions with regard to replication, integration, excision and transcriptional mechanisms. In some instances, the AAV used in this application is AAV9.

### Methods of Use

Described herein is a method for integrating an exogenous sequence into a chromosomal sequence of a eukaryotic cell, the method comprising: a. introducing into the eukaryotic cell: (i) at least one RNA-guided endonuclease comprising at least one nuclear localization signal or nucleic acid encoding at least one RNA-guided endonuclease comprising at least one nuclear localization signal, (ii) at least one guide RNA or a DNA encoding at least one guide RNA, and (iii) at least one donor polynucleotide comprising the exogenous sequence; b. generating a double-stranded break a target site in the chromosomal sequence, wherein at least one guide RNA guides the at least one RNA-guided endonuclease to the target site; and c. repairing the double strand break using a DNA repair process, thereby integrating the exogenous sequence into the chromosomal sequence of the eukaryotic cell, wherein the efficiency of integrating the exogenous sequence is about 20%, 25%, 30%, 35%, 40%, or 45% higher compared to a reference sample.

Suitable RNA-guided endonuclease are known in the art. For example, Journal of Hematology & Oncology volume 8, Article number: 31 (2015), and Genetics 2013 October; 195 (2): 303-308 describe RNA-guided nucleases for genome editing.

In some embodiments, the efficiency of integrating the exogenous sequence is about 5%, 10%, 15%, 20%, 25%, 30%, 35%, 40%, 45%, 50%, 55%, 60%, 65%, 70%, 75%, 80%, 85%, 90%, or higher. In some embodiments, the efficiency of integration is measured by the ratio between the cells that have integrated exogenous sequences and the cells without integrated exogenous sequences, or the total number of cells. In some embodiments, the efficiency is measured in cardiomyocytes.

In some embodiments, the efficiency of integrating the exogenous sequence is about 5%, 10%, 15%, 20%, 25%, 30%, 35%, 40%, 45%, 50%, 55%, 60%, 65%, 70%, 75%, 80%, 85%, 90%, or higher compared to a reference sample.

In some embodiments, the reference sample is a cell, e.g., a eukaryotic cell, whose genome has been modified using a different method than the methods described herein. In some embodiments, the reference sample is a cell, e.g., a eukaryotic cell, whose genome has been modified using a different CRISPR/Cas9 system. In some embodiments, the reference sample is a cell, e.g., a eukaryotic cell, whose genome has been modified using a different AAV/CRISPR/CAS system. In some embodiments, the reference sample is a cell, e.g., a eukaryotic cell, whose genome has been modified using a different AAV/CRISPR/CAS system with the same integrated exogenous sequence.

In some embodiments, the reference sample is a cell, e.g., a eukaryotic cell, whose genome has been modified using a different AAV/CRISPR/CAS system with the same integrated exogenous sequence at the same genetic locus, e.g., Mb or Des. In some embodiments, the reference sample is a cell, e.g., a eukaryotic cell, whose genome has been modified using a different AAV/CRISPR/CAS system with the same integrated exogenous sequence at a different genetic locus, e.g., a locus other than Mb or Des, or a locus at Mb or Des but at a different position as the one used in the methods described herein.

In some embodiments, the exogenous sequence is integrated into the 5′ or 3′ of Mb. In some embodiments, the exogenous sequence is integrated into the 5′ or 3′ of Des.

The compositions and methods described herein can be used to treat one or more disease or disorder associated the loci described herein. In some embodiments, the disease or disorder is a cardiomyopathy. In some embodiments, the disease or disorder is familial hypertrophic. In some embodiments, the disease or disorder is congenital fiber-type disproportion. Any other disease or disorder that can be treated by target the loci described herein can also be treated by the methods described herein.

Any suitable exogenous gene can be used in the methods described herein for transgene expression. In some embodiments, the exogenous sequence contains one or more mutations (or correction of mutations) of a gene that relates to the disease being treated.

The adeno-associated viruses are one of the most common tools for transgene delivery. The AAVs are part of the parvovirus family and consist of a single stranded DNA virus and have a packaging capacity of about 4.7 kb. Their main advantage is their low immunogenicity and the property that they remain episomal, therefore causing a low risk of mutagenesis. The episomal nature of the recombinant genome does make it sensitive to dilution via cell division (see, e.g., Davidsson, M., Negrini, M., Hauser, S. et al. Sci Rep 10, 21532 (2020)).

The novel HDR-based gene editing systems described herein avoid the problem of viral vector dilution (see, Example 2). Therefore, the systems described herein are particularly useful for skeletal muscle diseases (e.g. Duchenne), since viral dilution is more problematic for skeletal muscle disease.

In addition, the systems described herein are also useful for applications such as fetal or neonatal AAV gene therapy, which have been challenging due to problems caused by viral dilution.

It is further appreciated that—while the examples below demonstrate that a fluorescent protein (mScarlet) can be used to achieve integration—many other genes can be integrated depending on interest to express the gene. For instance, a mutation in a gene in a subject can be identified. Using the system and loci provided herein, a wild-type (e.g., non-mutation-containing) sequence can be inserted at any one of the loci (e.g., at Mb or Des) provided herein.

The terms “treat” or “treating,” as used herein, refers to alleviating, inhibiting, or ameliorating the disease or infection from which the subject (e.g., human) is suffering (e.g., a cardiomyopathy). In some instances, the subject is an animal. In some embodiments, the subject is a mammal such as a non-primate (e.g., cow, pig, horse, cat, dog, rat, etc.) or a primate (e.g., monkey or human). In some instances, the subject is a domesticated animal (e.g., a dog or cat). In some instances, the subject is a bat. In some instances, the subject is a human. In certain embodiments, such terms refer to a non-human animal (e.g., a non-human animal such as a pig, horse, cow, cat or dog). In some embodiments, such terms refer to a pet or farm animal. In some embodiments, such terms refer to a human.

The compositions can be formulated or adapted for administration by injection (e.g., intravenously, intra-arterial, subdermally, intraperitoneally, intramuscularly, and/or subcutaneously); and/or for transmucosal administration, and/or topical administration. In some instances, the administration is subcutaneous. In some instances, the administration is intravenous.

An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments. For example, effective amounts can be administered at least once.

### EXAMPLES

The invention is further described in the following examples, which do not limit the scope of the invention described in the claims.

### Example 1: Efficient In Vivo Homology-Directed Repair Within Cardiomyocytes

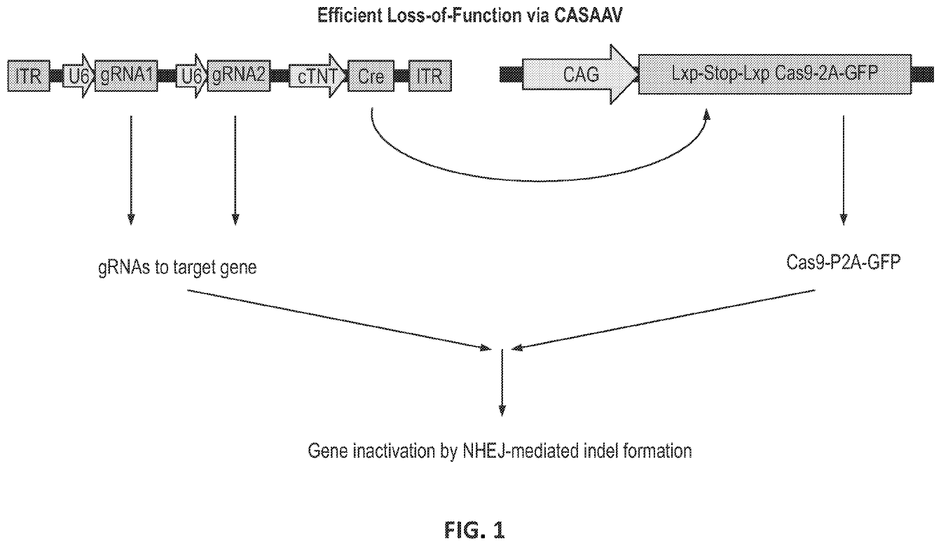

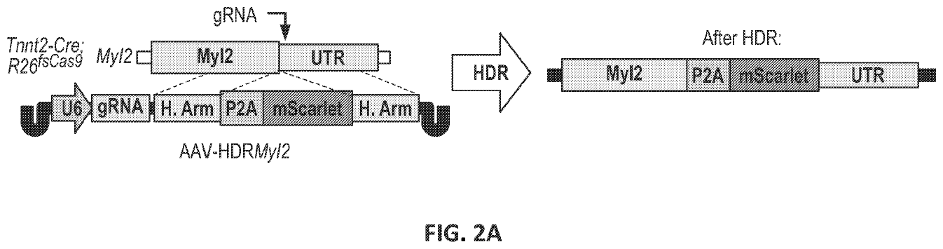

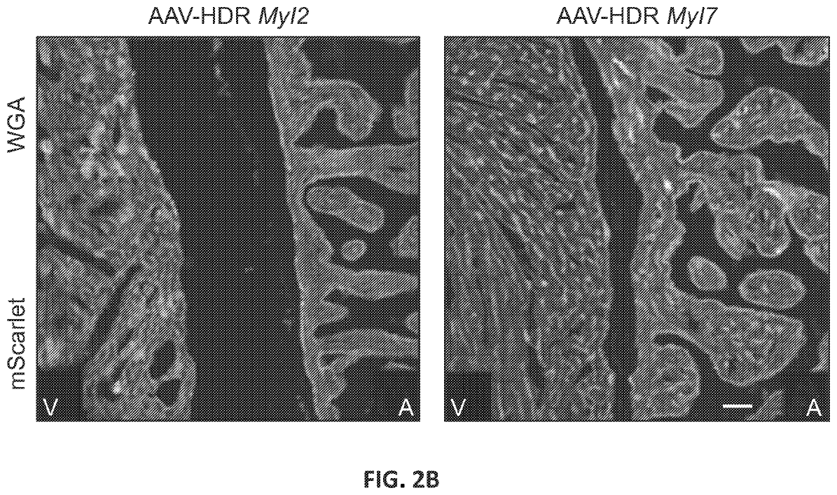

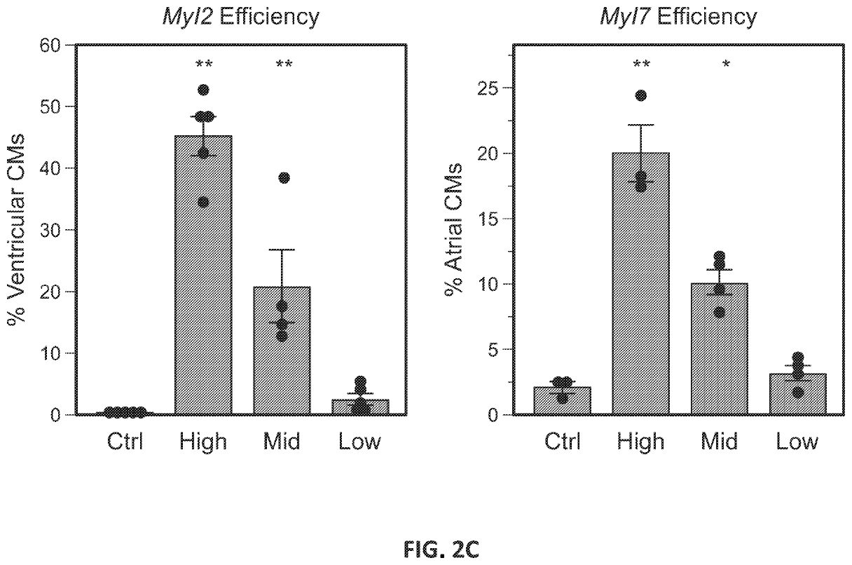

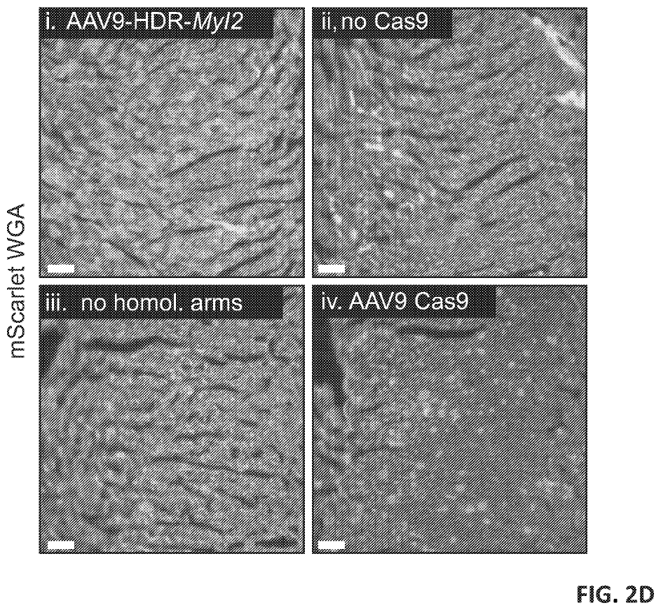

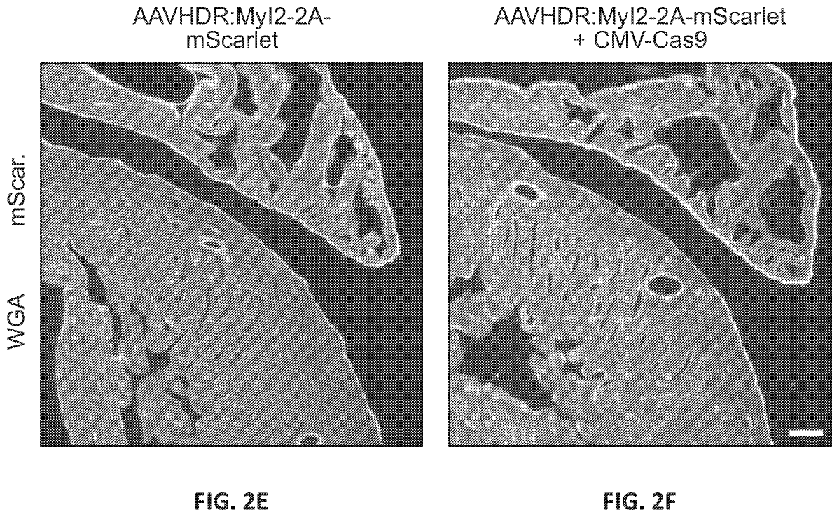

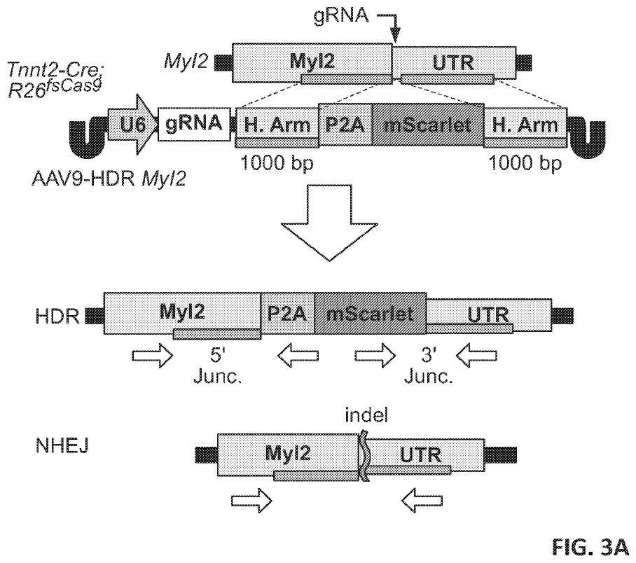

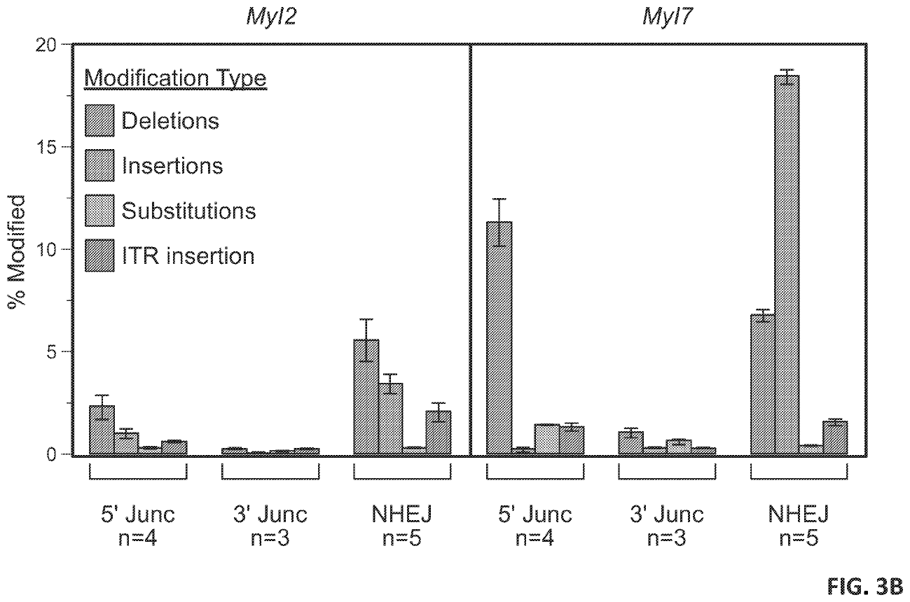

Here, we studied CASAAV-HDR in cardiomyocytes and skeletal muscle cells. FIG. 1 is an example showing the mechanism for efficient loss-of-function via a AAV and Cas9 mediated double strand breaks followed by non-homologous end joining. When a DNA template with regions of homology 5′ and 3′ to the double strand break is available, homology directed repair can repair the break using the template and accurately incorporate the sequence between the homology arms (FIGS. 2A and 3A).

We constructed an AAV9 vector containing a gRNA sequence targeting (i.e., complementary to) a ventricle specific gene: Myl2. The AAV9 construct further included a promoterless HDR template that replaces the native stop codon with self-cleaving 2A peptide followed by mScarlet, a red fluorescent protein (FIGS. 2A and 3A). The vector was injected subcutaneously into Cas9 expressing newborn mice. Briefly, we observed mScarlet expression within a remarkably high fraction of cardiomyocytes, approximately 45%. Expression was ventricle specific, consistent with the Myl2 expression profile. Similarly, when we targeted an atrial specific gene (i.e., Myl7), we observed mScarlet expression in ˜20% of atrial cardiomyocytes. More particularly, subcutaneous delivery of the AAV to postnatal day 0 (P0) mice with cardiac-restricted Cas9 expression (Tnnt2Cre::Rosa26fsCas9) yielded strong mScarlet expression in P7 ventricular cardiomyocytes (FIGS. 2B and 2D). mScarlet expression required Cas9 and Myl2 homology arms (FIG. 2D ii-iii). AAV-delivered Cas9 successfully directed mScarlet expression. Guide RNAs targeting sequences on either side of the stop codon had equivalent performance (data not shown). We did not detect mScarlet in atrial cardiomyocytes, consistent with the targeted Myl2 allele retaining its expression pattern (FIG. 2B, left, and FIGS. 2E-2F). Parallel experiments targeting atrial specific My/7 (aka MLC2a) resulted in mScarlet expression within atrial but not ventricular cardiomyocytes (FIG. 2B, right). Flow cytometry quantification of My/2 knock-in efficiency after systemic AAV injection showed that ˜45% of ventricular CMs displayed strong fluorescence, with steep dose response (FIG. 2C). In atrial cardiomyocytes, My/2 knock-in efficiency was 20% and had similar dose-response (FIG. 2C). These data demonstrate proof of concept that integration at two specific loci—at Myl2 and Myl7—result in cardio-specific expression of the inserted gene (here: mScarlet).

Next, we quantified mutations created during the CASAAV-HDR DNA repair process (FIG. 3B). Amplicon sequencing of Myl2 and My/7 transcripts showed that the vast majority of transcripts with an insertion were mutation-free, indicating that CASAAV-HDR is precise without introduction of mutations. Furthermore, CASAAV-HDR efficiency was comparable when AAV was delivered to fetal, neonatal, or mature mice. In particular, the 5′ and 3′ junctions between the inserted template and the endogenous Myl2 or Myl7 sequences were amplified from RNA and deeply sequenced. We also quantified mutations in alleles that did not contain an inserted template by amplifying and sequencing cardiomyocyte genomic DNA flanking the gRNA target site. For Myl2, 95.9% and 99.4% of transcripts containing the inserted template had the expected 5′ or 3′ junction sequences, respectively, while 11.3% of alleles lacking an insert contained a mutation, reflecting non-homologous end joining (NHEJ) (FIG. 3B). For Myl7, these numbers were 85.8%, 97.8%, and 27.1%, respectively (FIG. 3B). These data indicate that CASAAV-HDR insertion is precise, and that a subset of alleles without repair template integration contained NHEJ-induced mutations. Integration of AAV inverted terminal repeats (ITRs) at Myl2 or Myl7 was detected in less than 2% of sequences (FIG. 3B). ITR-seq found ITR integration elsewhere in the genome 4-28 fold less frequently than at Myl2. Although HDR has been thought to be limited to proliferating cells, CASAAV-HDR occurred in postmitotic neurons and in postmitotic adult cardiomyocytes.

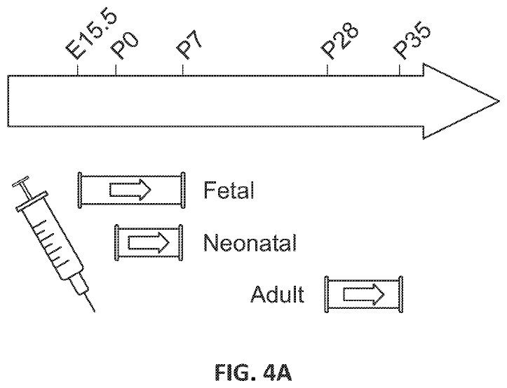

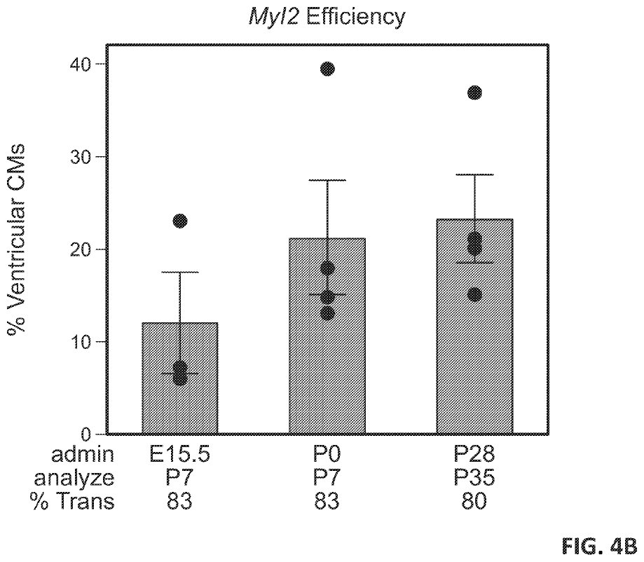

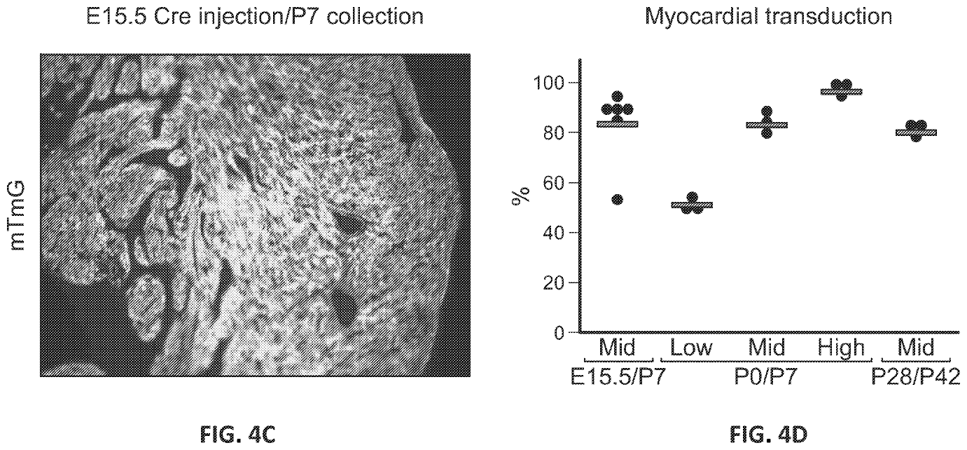

We then assessed the effect of cardiomyocyte proliferation on CASAAV-HDR efficiency by measuring CASAAV-HDR at Myl2 at different developmental stages. The experimental timelines for injections in fetal, neonatal and mature mice are shown in FIG. 4A. Surprisingly, CASAAV-HDR efficiency was comparable when AAV was delivered to fetal, neonatal, or mature mice (FIG. 4B). In independent pilot experiments, we established that AAV injection at these different stages yielded roughly comparable transduction efficiency. In these experiments, AAV-Cre was delivered to Rosa26^{mTmG} mice, in which Cre replaces expression of RFP with GFP. We found that the % GFP+ myocardial cells was similar with fetal, neonatal, or adult AAV delivery (FIGS. 4C-4D). Together, these data indicate that CASAAV-HDR occurs with comparable efficiency in post-mitotic and mitotic cardiomyocytes.

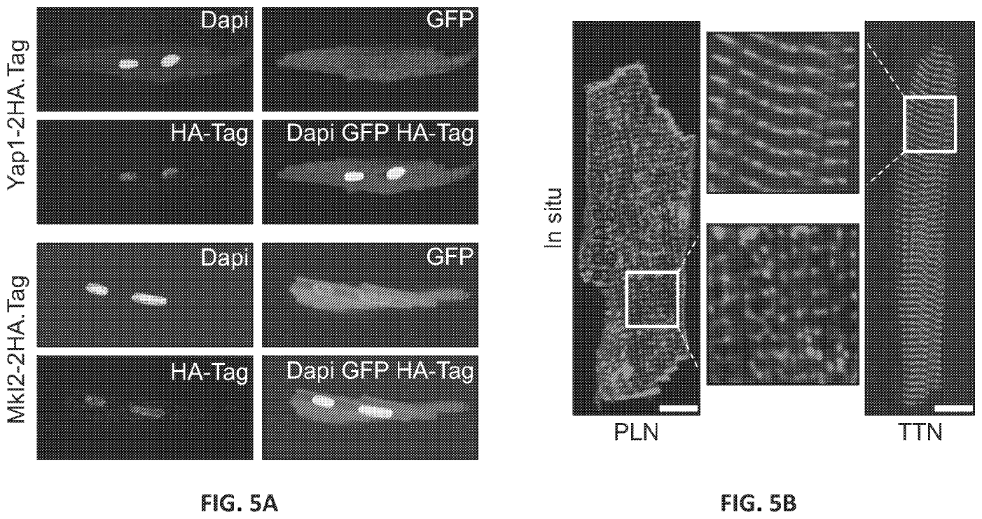

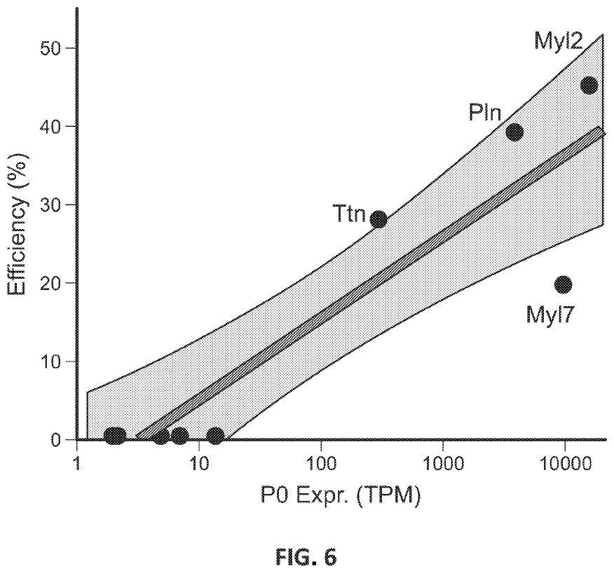

Next, we targeted seven additional loci: Yap1, Tmem43, Nfatc3, Bdh1, Mkl1, Ttn, and Pln, fusing either an HA tag or mScarlet to each. Representative images detecting the HA-tag for Yap1 and Mkl1 are shown in FIG. 5A. Overall, insertion efficiency varied dramatically between loci, with HDR efficiency generally correlating with target gene expression (FIG. 6). TTN-mScarlet and mScarlet-PLN fusion proteins localized to the sarcomere and sarcoplasmic reticulum, respectively, consistent with the localization of the endogenous proteins. The six lowly expressed genes (TPM<5) had low HDR efficiency, while the four robustly expressed genes (TPM>100) had HDR efficiency ≥20% (FIG. 6). TTN-mScarlet and mScarlet-PLN fusion proteins localized to the sarcomere and sarcoplasmic reticulum, respectively, consistent with the localization of the endogenous proteins (FIG. 5B).

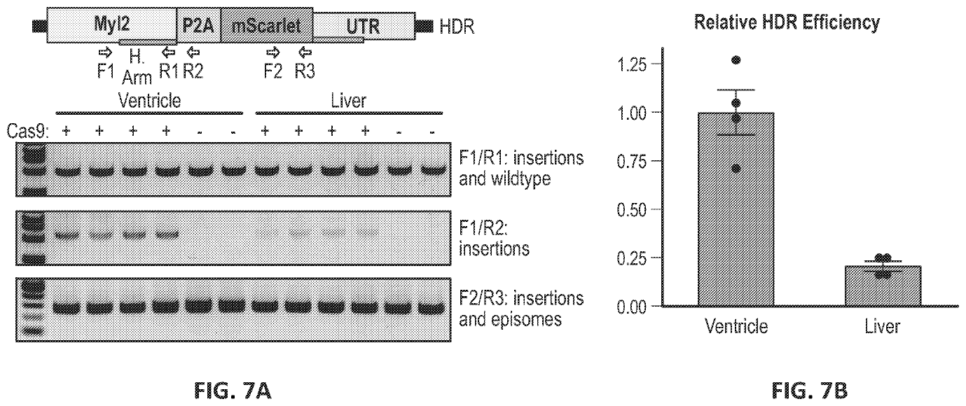

We investigated the frequency that HDR occurs in tissues in which the targeted gene is not expressed. For example, we investigated mScarlet integration at the Myl2 locus in liver cells (non-expressing) compared to ventricular heart muscle (robust expression). In this experiment, Cas9 was expressed in both cell types. We detected targeted insertion using specific primers (FIG. 7A, F1/R2) compared to primers that detect the native unmodified gene (FIG. 7A, F2/R3). We found that HDR was much more efficient in ventricle compared to liver (FIG. 7B). These data show that the level of expression of a gene and likely its chromatin state determines the efficiency with which it can be modified by HDR.

Collectively these data indicate that systemic delivery of CASAAV-HDR vectors can achieve efficient, precise, in vivo somatic genome modification that does not require cardiomyocyte proliferation. We successfully used this technology to monitor protein localization and anticipate it will be useful for many other applications, such as precise introduction of mutations to model disease or probe gene function. CASAAV-HDR may also enable efficient, permanent, and precisely targeted delivery of therapeutic transgenes to validated loci.

Systemic delivery of CASAAV-HDR vectors achieved efficient, precise, in vivo somatic genome modification that did not require cardiomyocyte proliferation. Efficiency correlated with expression level of the target gene and in the best case reached remarkably high levels (45%). To our knowledge, this work provides the first instance of successful systemic delivery with such a high level of HDR efficiency.

### Example 2: AAV-HDR System Avoids Vector Dilution

FIG. 8 is a panel of microscopic images showing the results of co-injection of AAV-CAG-Luciferase (luciferase) and AAV-HDR-Des-MS at P3 (mScarlet), examined at 18 days or 3 months after injection. Reduction of luciferase expression in skeletal muscle was observed at 3 months, compared to 18 days. In contrast, for HDR the mScarlet signal was consistent between time points.

### Example 3: Therapeutic Expression of Transgenes at the Myl2 Locus

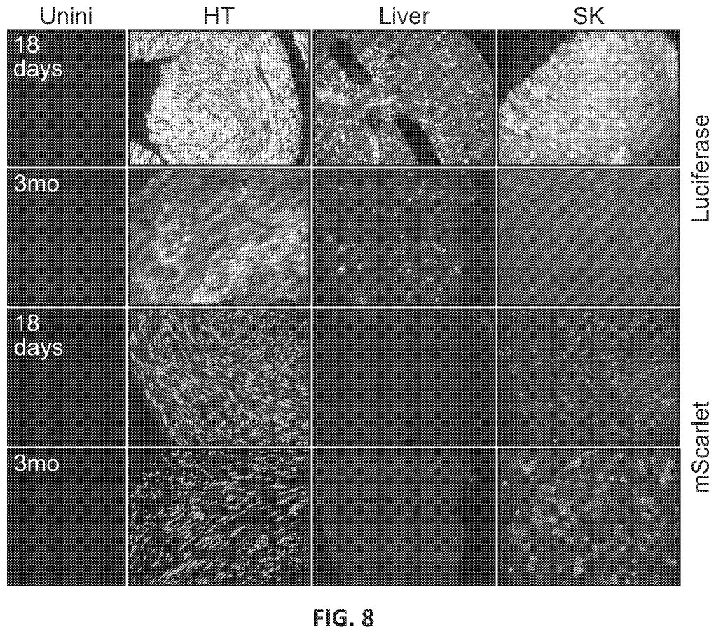

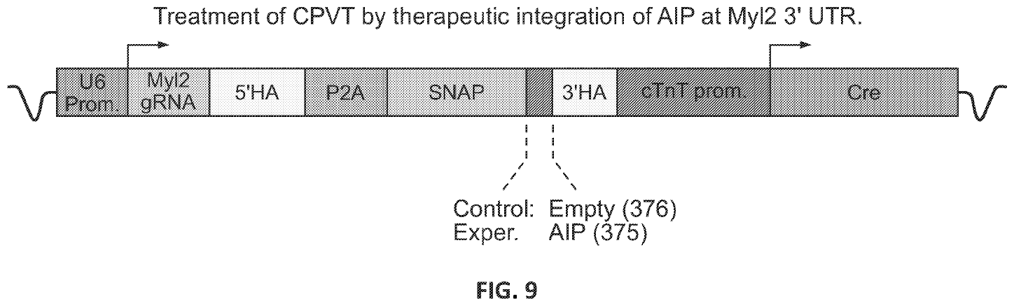

As shown in FIG. 9, a vector containing U6-sgRNA targeting Myl2 in 3′ UTR just after stop codon was generated. The HDR template was the 5′ homology arm (500 bp). SNAP is the imaging protein (https://www.neb.com/applications/cellular-analysis/cell-imaging/snap-cell) and AIP is a CaMKII inhibitor peptide (Bezzerides, Circulation. 2019; 140:405-419). The 3′ homology arm is about 500 bp. TNT-Cre was used to activate Rosa26-fsCas9-P2A-GFP in cardiomyocytes.

As for the therapeutic indication, CPVT refers to catecholaminergic polymorphic ventricular tachycardia. This is an inherited potentially fatal arrhythmia. It has been shown that it can be treated by AIP gene therapy (Bezzerides, Circulation. 2019; 140:405-419). The mouse model is RYR2^{R4650I/+}.

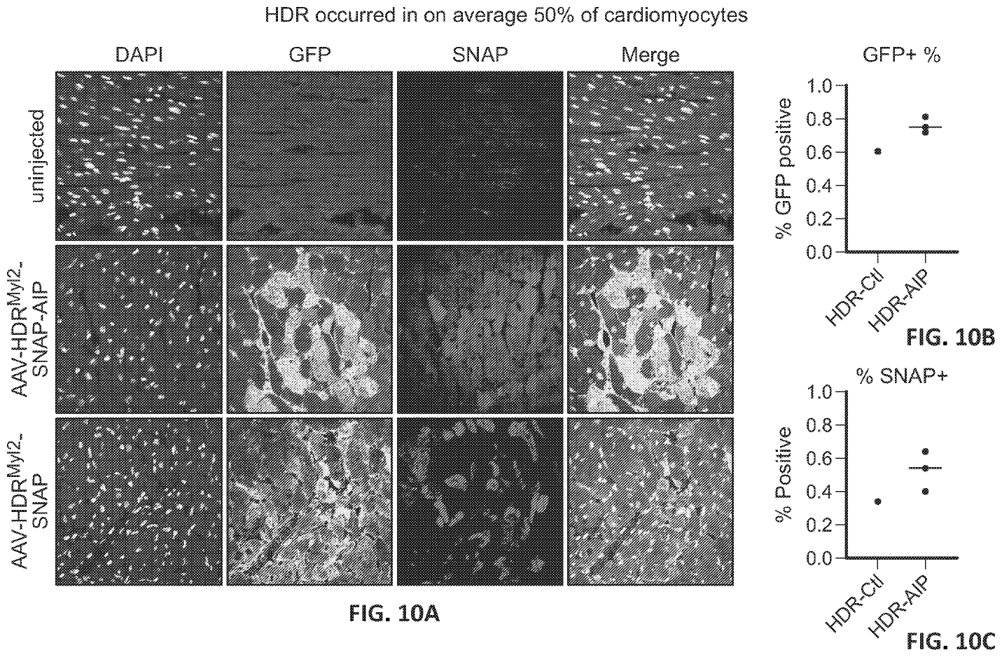

FIG. 10A shows that HDR occurred in on average 50% of cardiomyocytes. The GFP indicates AAV transduction and Cre recombination that activated Cas9-P2A-GFP. Since SNAP in AAV has no promoter, SNAP indicates HDR so that Myl2 regulatory elements drive SNAP or SNAP-AIP expression. In FIGS. 10A-10B, GFP+% indicates the transduction efficiency, and SNAP+% indicates the HDR efficiency.

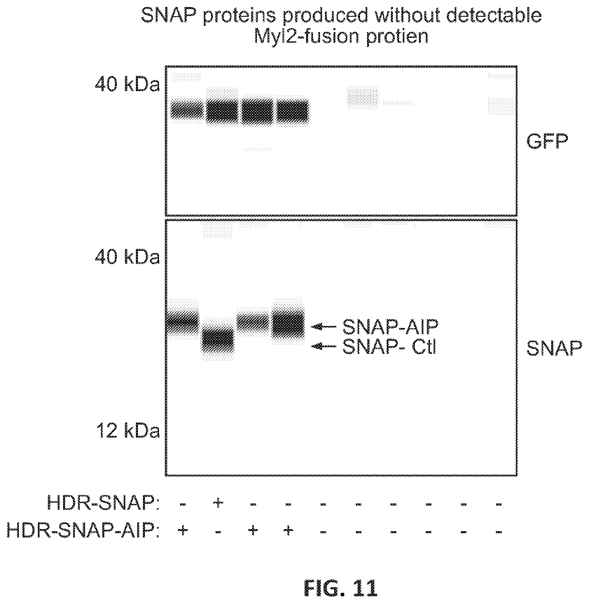

P2A can work imperfectly to yield fusion protein. HDR created Myl2-P2A-SNAP or Myl2-P2A-SNAP-AIP in this experiment. As FIG. 11 shows, SNAP proteins were produced without detectable Myl2-fusion protein. Capillary western for SNAP (20 kDa) did not detect fusion with Myl2 (a fusion should be ˜40 kDa, since Myl2 is 19 kDa). The bands for GFP indicate AAV transduction, with Cre expressed from AAV activating Cas9-P2A-GFP from the mouse genome. Mice treated with AAV9-HDR [Myl2]-SNAP were labeled as HDRMyl2-SNAP and mice treated with AAV9-HDR [Myl2]-SNAP-AIP were labeled as HDRMyl2-SNAP-AIP. The mice used were Ryr2^{R4650I/+}; Rosa26^{fsCas9-P2A-GFP/+}.

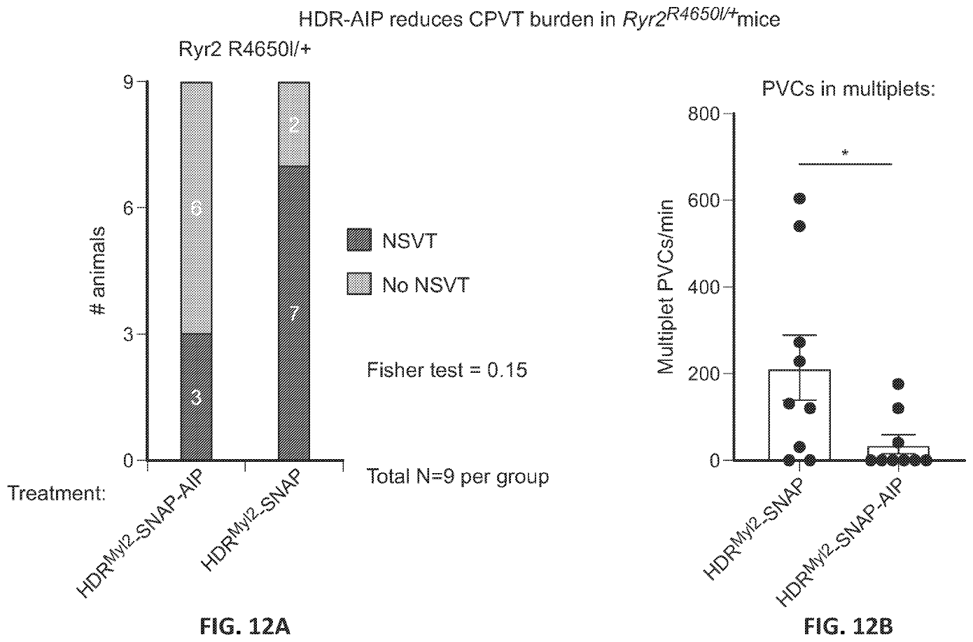

FIGS. 12A-12B show that HDR-AIP reduces CPVT burden in Ryr2^{R4650I+} mice. Specifically, CPVT mice were stimulated by injection with caffeine and epinephrine, and surface EKGs were recorded. NSVT is non-sustained VT, defined as 3 or more PVCs in a row.

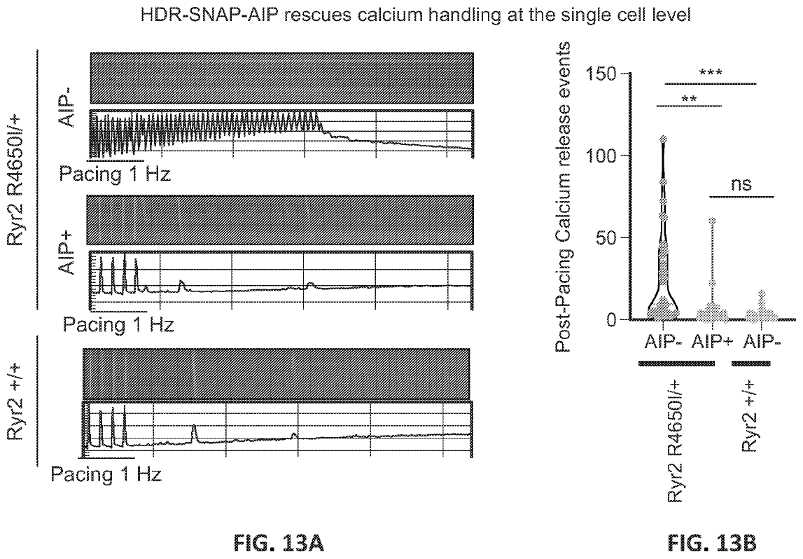

FIGS. 13A-13B show that HDR^{Myl2}-SNAP-AIP suppresses arrhythmias in CPVT mice. Specifically, individual cardiomyocytes were dissociated from CPVT mice treated with HDR^{Myl2}-SNAP or HDR^{Myl2}-SNAP-AIP. They were loaded with Ca2+ sensitive dye X-Rhod and electrically paced at 1 Hz. At cessation of pacing, normal cardiomyocytes are nearly devoid of Ca2+ oscillations for over 30 seconds. In contrast, CPVT cardiomyocytes with control treatment showed a high frequency of calcium waves, which were suppressed by AIP.

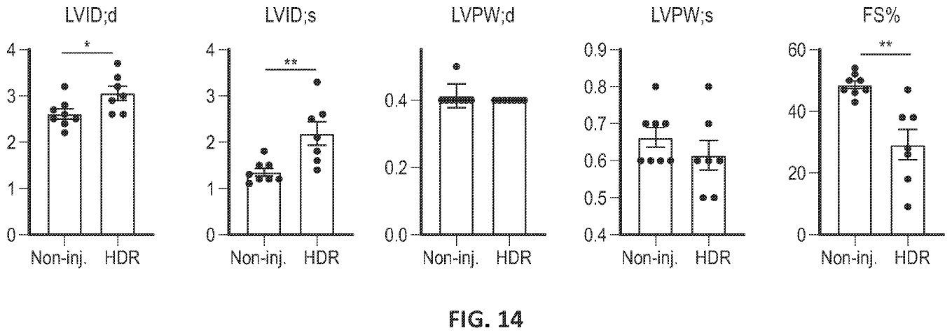

In FIG. 14, the effect on systolic ventricular function was tested. Specifically, control mice were treated with AAV vector that induces HDR at Myl2 (HDR^{Myl2}-mScarlet).

### Example 4: Additional Loci for High-Efficiency HDR Targeting

In this example, we tested several additional loci for HDR in mouse heart and identified several that support high efficiency HDR. We then checked heart systolic function by echocardiography. Two promising loci supported high efficiency HDR and did not cause ventricular dysfunction—myoglobin and desmin.

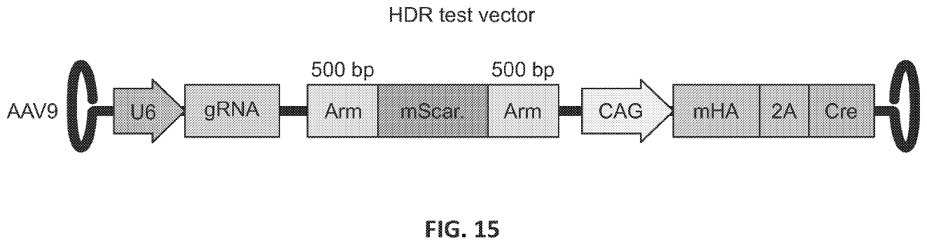

We set out to find loci that undergo efficient HDR and do not negatively impact heart function. The HDR test vector depicted in FIG. 15 contains (1) a U6 promoter driving a gRNA that cuts within the 3′ UTR of a candidate gene, near the stop codon; (2) an HDR template, comprising a 500 bp homology arm (sequence upstream of the gRNA cut site), mScarlet (red fluorescent protein), and a 500 bp homology arm (sequence downstream of the gRNA cut site); and (3) a CAG-mHA-P2A-Cre cassette, in which mHA is membrane bound HA that can be used to visualized Cre-expressing cells. AAV dose was 5E11/newborn (P0) mouse pup (about 2 g). Hearts were analyzed at p21. Mice were Rosa26fsCas9-P2A-GFP/fsCas9-P2A-GFP.

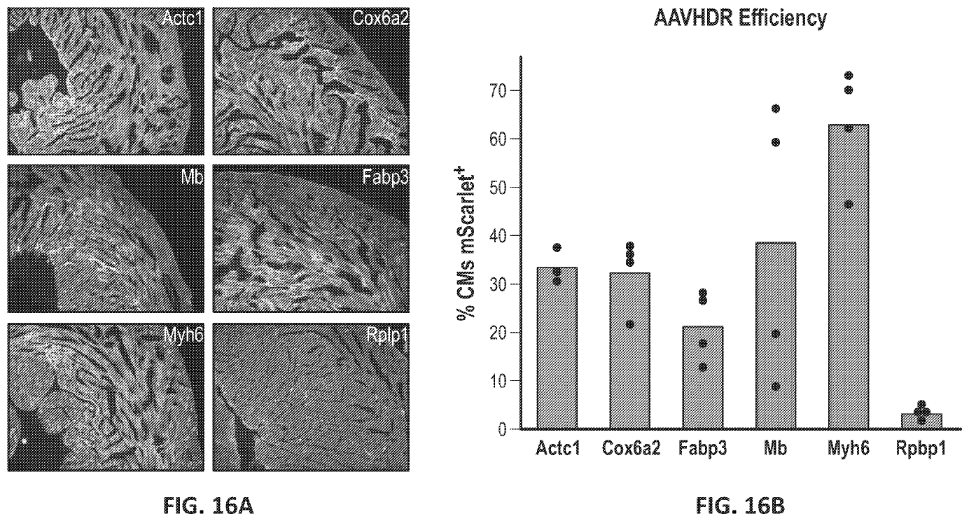

HA staining was performed to confirm that all viruses efficiently transduced hearts. % CMs mScarlet+ was measured for HDR efficiency. As shown in FIGS. 16A-16B, Myh6 was most efficient, followed by Mb.

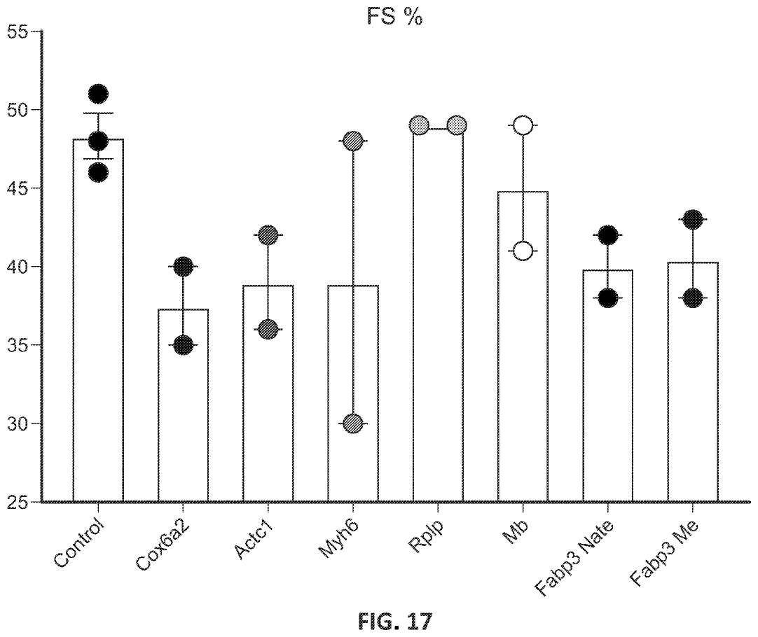

Echo was used to measure heart function. As shown in FIG. 17, Myh6, Actc1, and Cox6a2 HDR negatively impacted heart function, while FABP3, Mb, and Rplp did not. However, FABP3 and Rplp1's HDR efficiency was low. Based on these data, Mb appears optimal in terms of both HDR efficiency and safety.

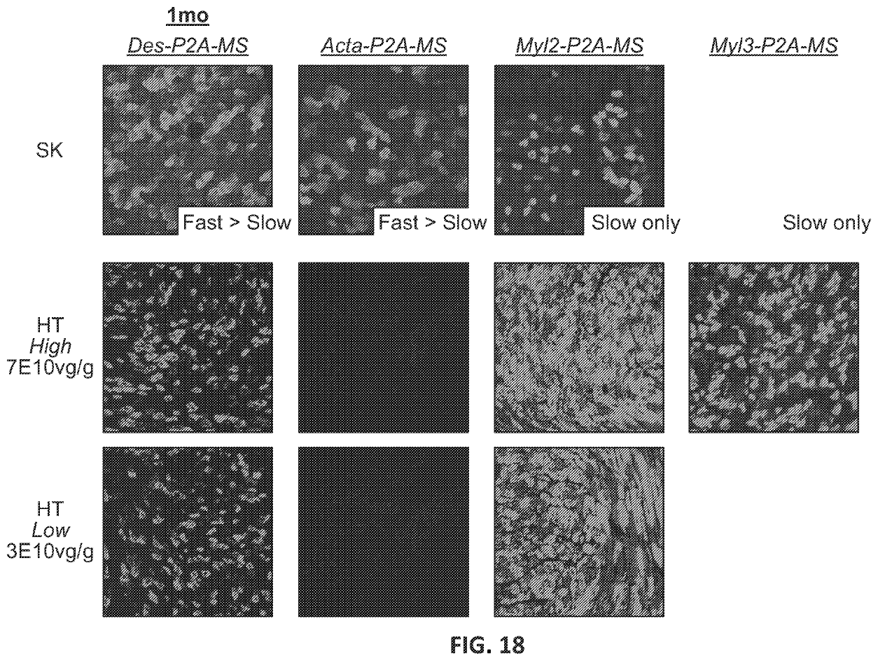

Based on their high expression in heart and skeletal muscle, we also tested additional candidate loci for HDR to express transgenes in these muscles: Desmin, Myl3, Acta1. FIG. 18 shows that Myl3 was comparable to desmin in HDR efficiency in heart, less than Myl2. Desmin was best of these in skeleton muscle and moderately efficient in heart.

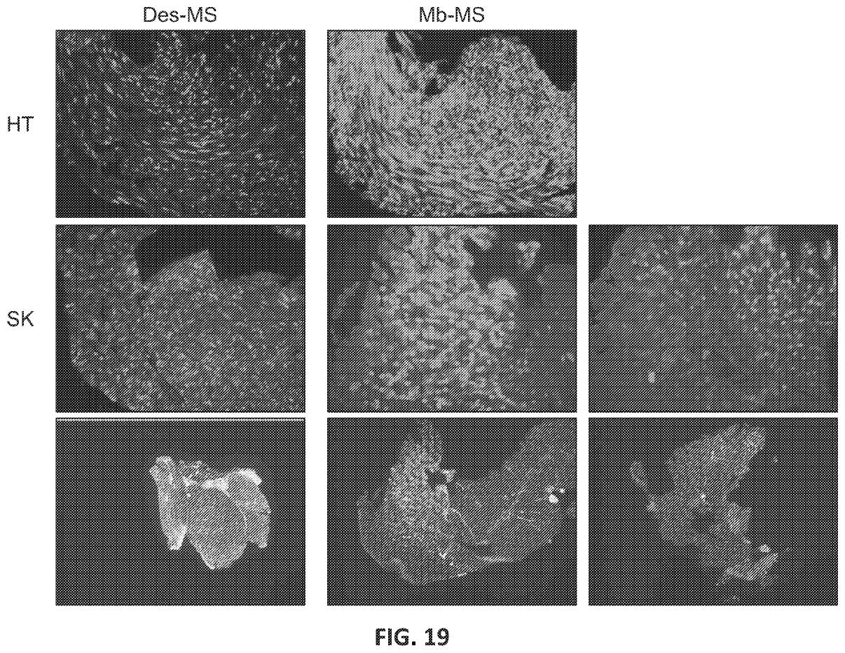

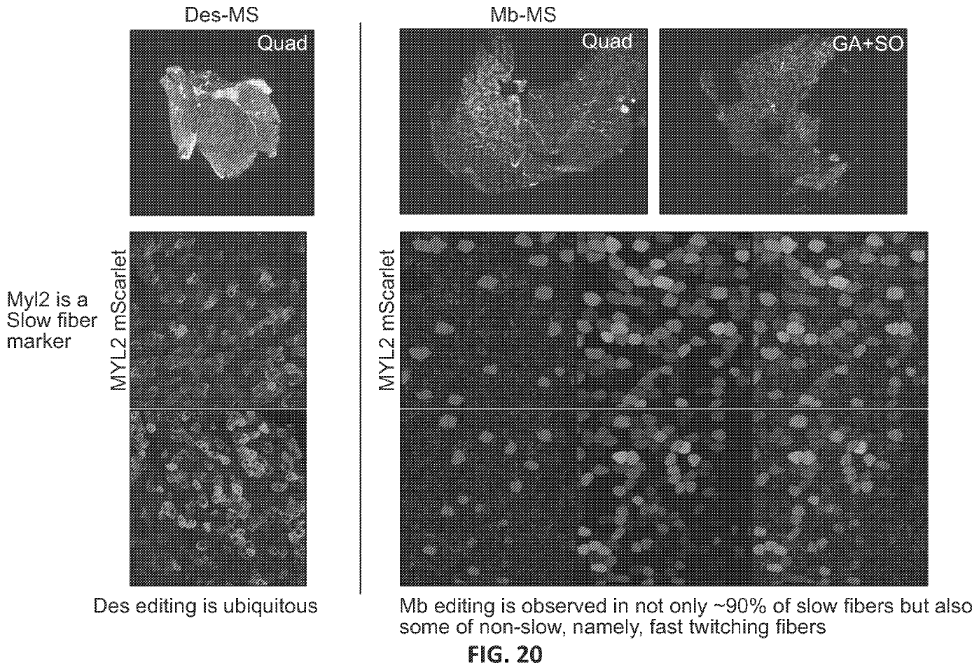

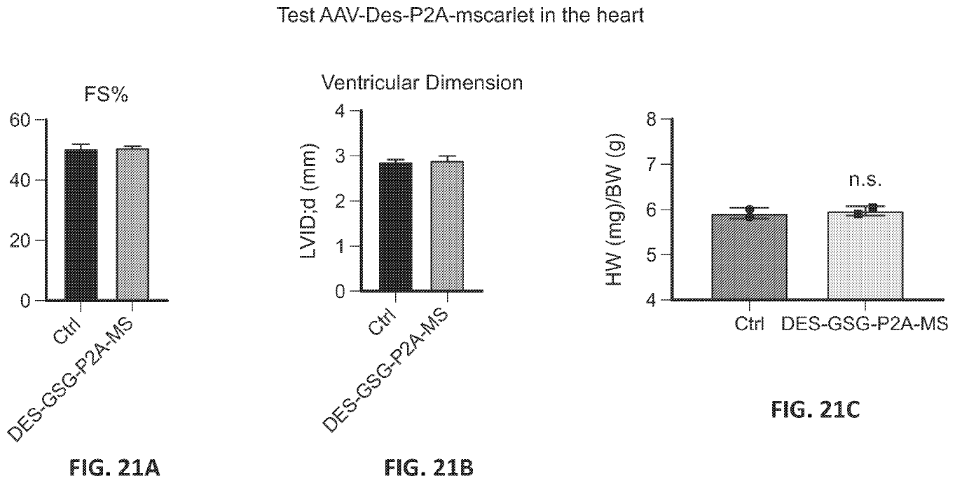

As shown in FIG. 19, compared to Desmin, HDR at the Mb locus was more efficient. As shown in FIG. 20, desmin HDR occurred in both slow and fast fibers, whereas for Mb, most slow fibers were edited. In addition, some non-slow fibers were also edited. Furthermore, FIGS. 21A-21C show that desmin HDR did not cause cardiac toxicity. Desmin targeting in heart does not affect ventricular size or function.

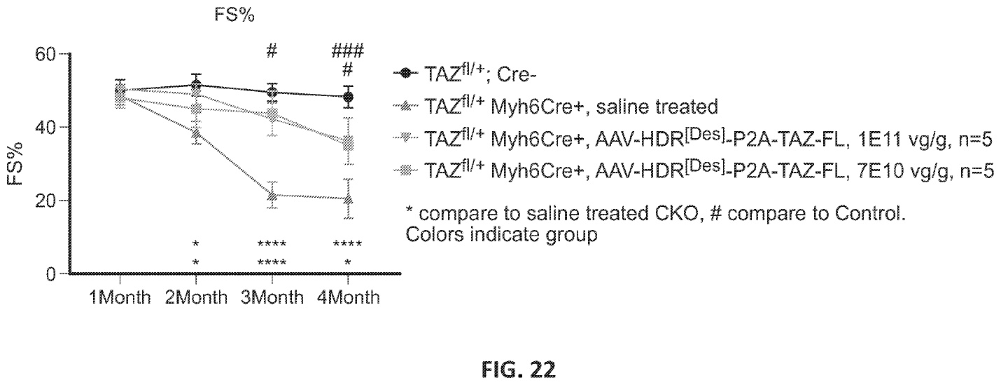

FIG. 22 shows the therapeutic efficacy of HDR-mediated editing: “Permanent” gene therapy for Barth syndrome. Barth syndrome is an X-linked cardiac and skeletal myopathy due to mutation of the gene Tafazzin (Taz). Mice with Taz mutation develop progressive cardiomyopathy by 3 months old. We treated new born TAZ cardiac-specific knockout mice with a vector that integrates P2A-TAZ at the desmin stop codon. Low and high dose administration partially rescued TAZ deficiency. The incomplete protective effect likely reflects the deterioration of unedited cardiac cells. This points to the necessity of editing as many cardiac cells as possible in the treatment of BTHS.

### Example 5: The HDR Editing System can Drive Efficient Transgene Expression in Skeletal Muscle

We also examined HDR efficiency in skeletal muscle. AAV9 transduction of skeletal muscle is relatively inefficient compared to heart. MyoAAVs have much better skeletal (and cardiac muscle) transduction than AAV9. Therefore we looked at skeletal muscle HDR at Mb, with delivery by AAV9 or MyoAAV2A.

Here the vector was: U6-gRNA [Mb]-5′HA [Mb]-P2A-Halo-AIP-3′HA [Mb]-Tnnt2-Cre. The vector was delivered to newborn mice and experiments were done at about P30.

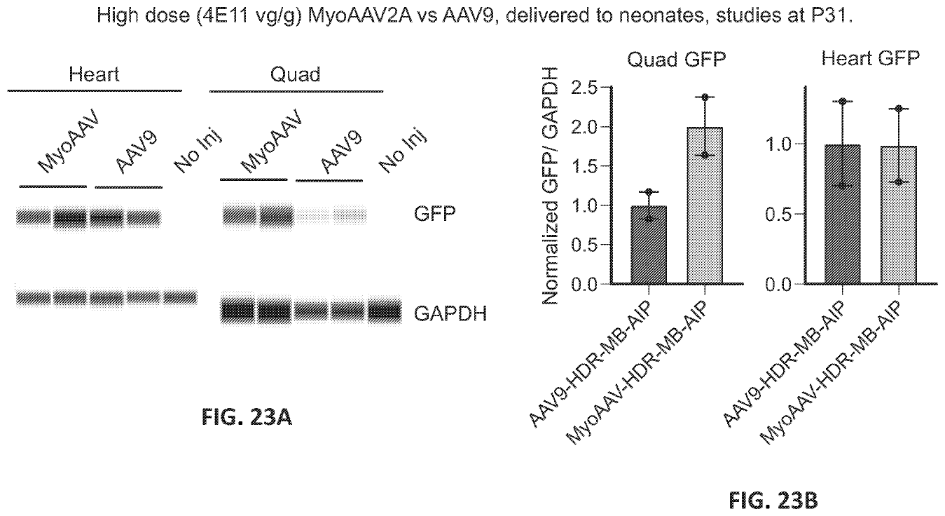

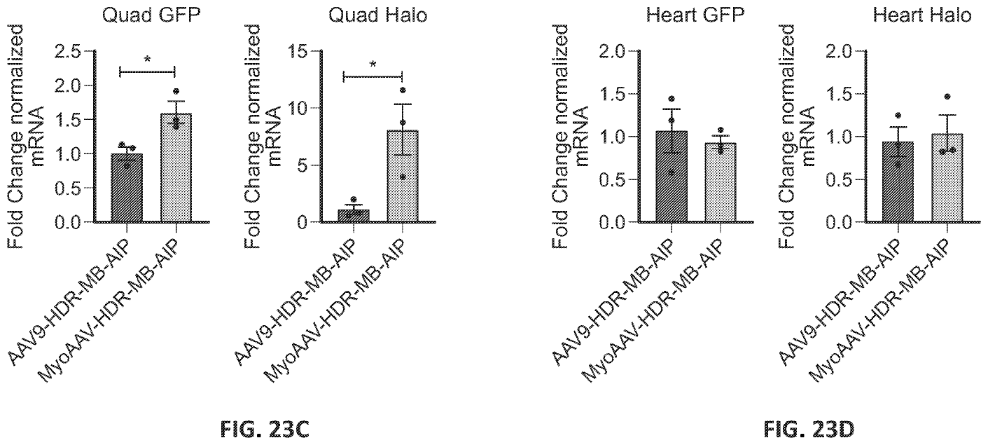

As shown in FIGS. 23A-23D, at this higher dose, cardiac MyoAAV has equivalent editing efficiency to AAV9 for heart. But MyoAAV2A is better for skeletal muscle. The GFP indicates transduction/Cre recombination (from Rosa26Cas9P2AGFP) and Halo indicates HDR. HDR appears to be more sensitive to transduction efficiency that GFP activation efficiency.

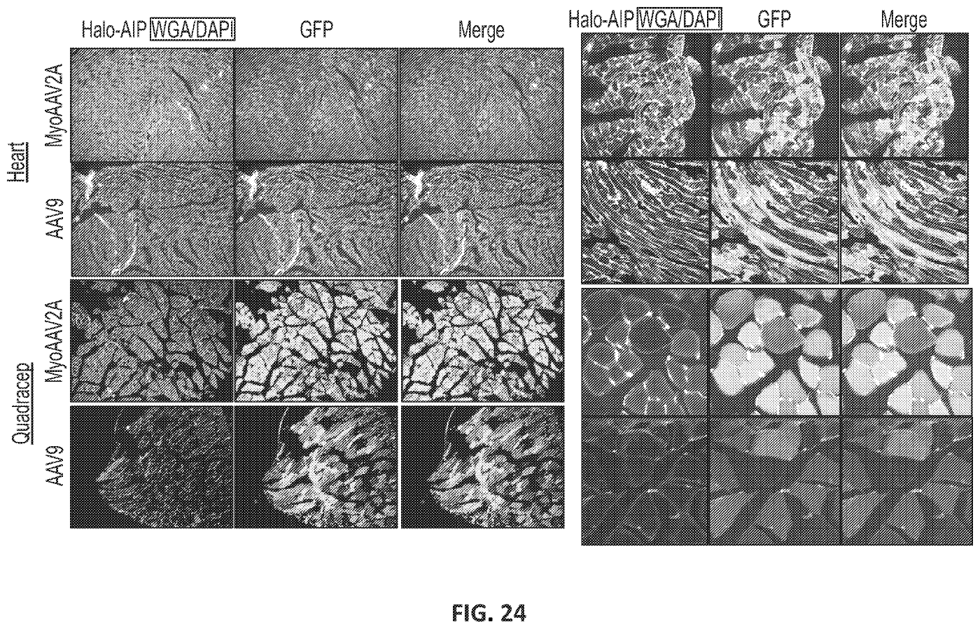

The same vector was delivered at a 40× lower dose (1E¹⁰ vg/g). At this dose, it is clear that MyoAAV2A was superior to AAV9 in both heart and skeletal muscle, for both transduction (GFP) and HDR (Halo) (see FIG. 24).

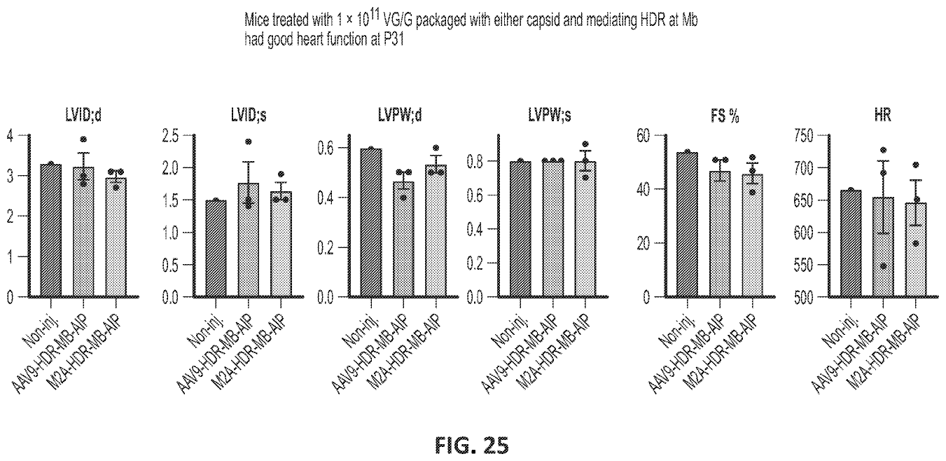

As shown in FIG. 25, mice treated with 1×10¹¹ VG/G packaged with either capsid and mediating HDR at Mb had good heart function at P31. Again, this demonstrates that HDR at Mb was well tolerated even with higher HDR efficiency supported by MyoAAV2A.

The guide RNA (gRNA) and homology arm (HA) sequences used in the above experiments are listed below:

Myl2
gRNA:
(SEQ ID NO: 1)
agtgggctgtgggtcacctg
5′ HA:
(SEQ ID NO: 2)
gtcacaggcgtgcattcagccacagtccccaccaagccctctcgcccctgagcatagccctgcccagcagctc
cttcaacagctggatctccattttcccctgctgggcatctgggaggttgcggggaggtctaggggacttcaga
tcaccccggtgagtaaaggtggggatggtgaagacacaagaacacgtggacccagaagttaatccaggacagt
agcctgggcttagttcccagagagaggtaacttttgggcctgctgacttccctcccacctcaagtcctgctga
cagcagtcacagcctggctgggggtattggggtccttcccaggatctcaggcccactctgcgccccccttgcc
attctgaggatctctgaatcccactactcttgcttgcagatcgaccagatgttcgcagcctttccccctgacg
tcaccggcaatcttgattataagaatttggtccacatcattacccacggagaagagaaagac
3′ HA:
(SEQ ID NO: 3)
gccctgaaccacaggatcaggtgacccacagcccactctccatcccagggctgtgcgcaaataaacaggaagt
cttggctctggctgtggtgaccattggctcatttggctctaagtagccaaagtcacaaagtatttgggttctg
tgaaggctgcagggatctcagtttgaacagcaaacaggcaccacctggcagtggtggcactttgtggggggca
cactccagccattgggagtcagaggcaggtggatctctgagttctaggctagcctggtctacagagtcagttc
caagaagtccacggacttcaggagcatctgcatccaggagtggcagttctgtcactctctccaggtggccttc
ccttagctctggttgctttctgggcccaggctttgaaggtgggaaaaaagacggatggatctctgggacttcc
agacttagcccgctttacatagtgggtttcaggctagcctgggctacataatgagatcctga
ACTC1
gRNA:
(SEQ ID NO: 4)
ggtcatcctgaatataaggt
5′ HA:
(SEQ ID NO: 5)
cacagatacggaactttaggactgcttctaaatagtgaataaaagcaaatatctgaaacacggtgtgtgtgtg
gggggggggggaagcaataactaaacctatctacacatttaaaaggtccccagcgtttgaactatccttcatg
aatattattctttagtacaaataagcagagaaatgatgaaatacaggaaggattgcaaagcctgattggctac
attgcttattgtatagcatctcctacgttgtctactatcggttaatctgtgatggtttttctgtgaatattcc
aagcctgttgcccacttcattgttggggacttcagaacacactgatggttttgttttctctctgcagattatt
gctccccctgagcgtaaatactctgtctggattgggggctccatcctggcctctctgtccaccttccagcaaa
tgtggatcagcaagcaagagtatgatgaggcaggcccatccattgtccaccgcaagtgcttc
3′ HA:
(SEQ ID NO: 6)
gatgtctctctcttagcataccttatattcaggatgaccgtattgtgcttcttggaatcctctgagccccctc
cccatctctcatcagtcattgtacagtttgtttacacaagtgcagtttgtttgtgcttcaaatatttattgct
ttataaataaaccagaccaggacttgcaacctacaaaagcctgtgttctttcttgtgtgagtgggcctgggat
ggagaaggtgttcactctgatactccgagcattacaatattagtgcaacacaaagtttacccaatgaattcat
gtggtgttgaattgagctaggagttacagaggtacatcttttgggatggagttatgaatatttttgaatctta
taagcaaagattagttctgtggtttagttgttaggtttagttctgtataagtaaggaatgaagatagaaaaca
caattatctacaaccagttgccatgtttggtactaaagttgctttagcacatatcacatatt
Cox6a2
gRNA:
(SEQ ID NO: 7)
caaattggccttctgcacac
5′ HA:
(SEQ ID NO: 8)
gggcaggaggtaagtggggccaggcctgactctgatttagtagcctagccgatctgttctctttgcccggctt
ccccctccttctgtctcctggctcctctctcctgatctaccaggcactgatctttcagacctctaggcccagg
gatagtttttgcctatgaatctactcacaccccacctttgctcctccagccaacacctggcgcctcctgacct
ttgtgctggctettcccggcgtagccctctgctcccttaactgctggatgcacgctggccaccacgagegccc
agagttcatcccgtatcaccacctccgcatccgaaccaaggtacgccagaggatgagcgagtgagagcatgca
gggggattgcggggagggactgtggtgggtgacctgtctgccttctctctgcagcccttcgcctggggggacg
gcaaccacacgcttttccacaatccccacgtcaatcctttgcccaccggttatgagcaccct
3′ HA:
(SEQ ID NO: 9)
tgtctcagcagacacgctctgccagcaatcttcaaattggccttctgcacaccagctctgagagcccctgagg
ttccagtggacagttccaagctcaataaaggtgtggaagttttgtgtcctctggctctttgggaacagcatgg
tggaaggggctgggcaggctcttgggcagttggtatctgggttccagttatttttttttatataaggaaaatg
tgatgtttccacctgcattttcattttattttttaaaaatttatacaagcaatacattgaatattgagcatat
tactcagtcttgtgttagttcttatccatatgtaaatttatgctgaccccattcctgctcttgcaaaaaaagt
ggatcagcactgaaatctgacacatgggcaaaccataaaaatctcccagctcaggaggtagggcatcatttct
gggaagtttcccaggctagcccagagctgaagagaacttgttcccatgccagtagattcata
Fabp3
gRNA:
(SEQ ID NO: 10)
ccagttggcagaggagcggg
5′ HA:
(SEQ ID NO: 11)
gaagggcctagctctggaagggaggtaacaaagaggatcatgttcactaggtagctagagtaaggtgaggcca
tggctgggctctacagtgccagaatactgcatgttaggggagggaggaaaagttggcagcttagcagtttctc
aaggctctgttcttatcacccgtttgtctgccacatacaccaggcacccccttaggcaggtgctgaaatgaac
acaaaggaggaacaggaatgtactggatcttgaccagtttacctcctctctcaagggcctatttttcccccaa
atctctaaaatgctaattataacatcttaaaagatttgtatcagaaaaaaaagtaaagtgcctggcacacagt
aggtgctcaagtgctggtcaggatgagggtggggagcactccctcctctgctctgccccatctgaaacctgtc
tttctttctagactctcactcatggcagtgtggtgagcactcggacttatgagaaggaggcg
3′ HA:
(SEQ ID NO: 12)
cctggctgctccgtcactgacagcccgctcctctgccaactggccacccctcagctcagcaccatgctgcctc
atggttttcccctctgacattttgtataaacattcttgggttgggatttttctggagatacggggcatcagcc
tggacccagttcctactatgtatgtggtttattttttaaaactgtatccaaagggtgctccaaggtcaataaa
gcagaaccaaggccacccagttgtctgtctttggtcctcctttcctgtgtgtcaggttgaaatgaaggcctat
aggtcacctgggaagcagcactgtcaaggagccgagtggacaggctcaaggctcagttagggaacagtagcac
ctatgtaatacccttacactgacctgccaaggctcagagaagctagctgtcattctagcatctatgcaagccc
ttacactggcctgcccatggcagagcagctggctgtcactgtgtggctatttcacattcatc
Myh6
gRNA:
(SEQ ID NO: 13)
tctccagcagaccctcgctg
5′ HA:
(SEQ ID NO: 14)
agcaggccaacaccaacctgtccaagttccgcaaggtgcagcacgagctggatgaggcggaggagagggcgga
catcgccgagtcccaggtcaacaagctgcgggccaagagccgggacattggtgccaaggtgagttcctccctt
ggaactgctagtcacgcgctcatcaggatgcccctgcaagcatgatgctctgaacagctacgtgtgtatccca
tgtgcatttagacatagaataggcatacatacaaccgaagggtcccacagacacctgtaacctgagagtccac
tctcccacttgaagtgggccccagatctgtgttctaaactacaagcactgatatcatgagacttcaagactgc
tgaggttcaacccctcccttcccaagggcattttattagagccactgaaatccctagaaattcctccccaggc
catcgtacccactgacctcacatctcttactcctttctcagcagaagatgcacgacgaggaa
3′ HA:
(SEQ ID NO: 15)
cctctccagcagaccctcgctgtagccaatccacaataaacataaacgttcgactctgcctgcaccctgtcct
tcctaccaagcgtctctctgggggggaattttggtggtccaggaggagggtggccacagcagggactgtcaga
gctgtaagatctgaagcaccccatggtctgtggaagccgagagagaagtgaggtgtcttgcctcaggcagcac
agctacagccccagctccacttggtcgttgtgcaagtatagaaagggaaactttccaaacgtgggggaaacaa
atccaggtgtgagggatggccaagcctcagatgggaggtggaggcaagaggcaggctcagctagaaggcaagt
ttaaagccagcctgggtagcgtgagagctcacctcaaagacaagaaaacaacaacccctcaatgagtccaaag
aacagaggcactgaaagcgagccaagtcagatccagggggatggaagtttgctgacgagct
Rplp 1
gRNA:
(SEQ ID NO: 16)
ctaaactgcttttgttaagt
5′ HA:
(SEQ ID NO: 17)
cttagtttgtacttattgatgtggcttttatgtcaggccttcacggtccatggcttgagaatgagttttgttg
cctcttaagtttgggcatttgaagcagtgagattcatgcaaagtggatagctacattctatttatacaaacag
ctttatttctggcaagtggggtcaatcaactggtggcagttgtatattgattccctgtagtcctgaacattgc
tttggtctcatgggtgaaaagagcttaattgaatcattaaatcttgcagcacaaaactaaaagccttttctat
gtctctgttgtaggctctggccaatgtcaacattgggagcctcatctgcaatgtaggggctggtgggcccgct
ccagcagctggagctgcgcctgctggtggtgctgctccatccactgccgccgccccagctgaggagaagaaag
tggaggcaaagaaggaagagtccgaggagtctgaagatgacatgggcttcggtctttttgac
3′ HA:
(SEQ ID NO: 18)
actgcttttgttaagttagctaataaagagctgaacctgtaggtggactggtctcacttgtaagccacaaagc
tcttggagtttcagagatggtagcaggtcccaaaaaaatgtcagggaaggagggtcttgaccaaagccaccat
gagttatataaataagacctgggtcaaaagaactagacgatctgggtagagaatgggtgggtctgtctccagg
aagccccttcaagcaactgttcagagagcctgtttagtgccagacagacaggctgagagctaactcttactgt
caagaataacaaaataacctctgggtctcccgatgttcatgtgtgaagaacttgacagttgttgaggatctca
gaagcatctagaagggccagagaacctaccagtccactggtgtaaaaccagcagcctaaaggattcagtcacg
cagcaaggcttggtgtgtaactacaacttcatgtagaaagtttaggacttggggctgacagt
Mb-NVD
gRNA:
(SEQ ID NO: 19)
cagcttggtgggctggacag
5′ HA:
(SEQ ID NO: 20)
ccggctgctgggcctgcatcctgaagtagtgtgggcacacgagaggtgagaaaggggccccagaggggctccc
tgagtttgagtgacccccctgactgcatgacttcatgcacacagctatacctgtctttgcctcagtttcccca
tttacagagcagggatggtggtggtgcctgcttccaggtagtcaggtttaaaggagttggttcatggtaattg
catggaatggcacttggtacatcgtaagtgtgcaaggaacagtcctatagtaaggagaaggtcagtgagtaca
cacccccttagctcatggcttgctccgtcccctgacctacaacctcttgtccctttcttgcagtttatctcag
aaattatcattgaagtcctgaagaagagacattccggggactttggagcagatgctcagggcgccatgagcaa
ggccctggagctcttccggaatgacattgccgccaagtacaaggagctaggcttccagggc
3′ HA:
(SEQ ID NO: 21)
gccatgggctccaactgtccagcccaccaagctgggacccagtgttgtgtagcaagtagcgtgtgcagtgttc
taggttagcagagaacagaagaggggagcatagtgtggcatccacccacacccctggggacagggctctgggc
agtgttaccctggagcccagaggtgcaaagtggccttcgtcagctctgccgggtcatgctcaggtctcctaag
tcccagtccattttcttctggttttgggaaaatctcttttccactgtcacatttgaccccaaatccaagtcac
tgactagcagaccctgacctttgggcgagatggagggttgcttagagggagtggagggtgaaaacggggcggt
gagcatcaagtctcccactgctcagcttcccgttgacccaccttgtctcaataaaatatcctgcgagtcctca
attcttgtctccgtccggtatatttttatcccttccggtaagggaaacatccacaaacgtcg
MB-SW
gRNA:
(SEQ ID NO: 22)
ggcttccagggctgagccat
5′ HA:
(SEQ ID NO: 23)
tgcaaagatgtcctgtgcctctgggtgcacttgtgtccctcggtgtccccttctgctgtgtcccactctcatc
tctgctgtgtcacttgcacataagggccccagcccaaaacagcagctgtacccccactcctgagctagcaggg
gtgtgtccacagtccctgggtctaatctcagcttactatctacctgctacatgacctcagcctggtcacttgg
tagtctcttcagtacagcagccaaagctgtacgggaccaaactctaagtcaatgacgataagtaagtgttatg
atagtttcatcgtgagcctttggattctgggtggtcccgatccccatctaggcaatgtcggtacctgatctcc
cctggcccctctccccttgttagtacctgttcctcagtcccaggtttagggataaaatcacatgatgggaaaa
gcctcctcccaagtgtcctgattgacagtcctggtgggctagccgatgctgagcaaggtatcatgggccggct
gctgggcctgcatcctgaagtagtgtgggcacacgagaggtgagaaaggggccccagaggggctccctgagtt
tgagtgaccctccctgactgcatgacttcatgcacacagctatacctgtctttgcctcagtttccccatttac
agagcagggatggtggtggtgcctgcttccaggtagtcaggtttaaaggagttggttcatggtaattgcatgg
aatggcacttggtacatcgtaagtgtgcaaggaacagtcctatagtaaggagaaggtcagtgagtacacaccc
ccttagctcatggcttgctccgtcccctgacctacaacctcttgtccctttcttgcagtttatctcagaaatt
atcattgaagtcctgaagaagagacattccggggactttggagcagatgctcagggcgccatgagcaaggccc
tggagctcttccggaatgacattgccgccaagtacaaggagctaggcttcaagagc
3′ HA:
(SEQ ID NO: 24)
gccatgagctcccactgtccagcccaccaagctgggacccagtgttgtgtagcaagtagcgtgtgcagtgttc
taggttagcagagaacagaagaggggagcatagtgtggcatccacccacacccctggggacagggctctgggc
agtgttaccctggagcccagaggtgcaaagtggccttcgtcagctctgccgggtcatgctcaggtctcctaag
tcccagtccattttcttctggttttgggaaaatctcttttccactgtcacatttgaccccaaatccaagtcac
tgactagcagaccctgacctttgggcgagatggagggttgcttagagggagtggagggtgaaaacggggcggt
gagcatcaagtctcccactgctcagcttcccgttgacccaccttgtctcaataaaatatcctgcgagtcctca
attcttgtctccgtccggtatatttttatcccttccggtaagggaaacatccacaaacgtcgctggaagaaat
gggaaggtgcattctgggatggcgggcagacagaagatgtgagacaggcccctttctacaatcacctctcaat
gacaagcacacactgggctaggttcagggacagggaagaaagggccccccacctgggctctgatcaagatggc
gggcctgcagacagggtcagctgaccttggcagtctgaggatcagtgccaggtgcccagggcaggagacaggg
aggagatgtctcgctgtggatccaacagctgaggcaatgtgagaaggtcacccagccagaacgtagcagagct
gagagccaaacagtggcctagaagacaactgggtctctgaccccaggacagagcctgtggccaacagtagcag
ctgctgaccttccggccagggagaggacagacaacagggagatactatccaaacacaggacaggggaagaaac
cca
Des-g2
gRNA:
(SEQ ID NO: 25)
gggccaggacactgaattcc
5′ HA:
(SEQ ID NO: 26)
ctagttgcttgctaagggactcatgagcgccaaactcctgacaccacggaactaaatgtgacccccactatga
aaactcagatttcttttttcacttatttgtttattcagataccaagtatgtgttctgcatataaaatcacttt
tgctgcggatcagcagctctgagacagaataagtggactatcatggctgctacttcccagagaggaaactgag
gcacaggagggactagctcctgcatcattaagacattaaacagctcatcctggagcctctaggttcctccagc
aggtctataggcctgggctttgaggtcctgtcttagatgagagtccacggatataaactcaccattcaggact
cttctgaaaatctggaggtggagagagagcaaggcaggataggaaacatgaatactgttcccgggccttgggt
cccctgctatcctgtaccaacacacagcacaatcttttggctcttgaagccattcctctagcaagactggtcc
ctctctctataaatcatctgttgggtgctgggttccaggatgaggtctcaaagaagtcagacagttgtcataa
gaaaggtgaaagtcagctagcacttggagcaatgcagcctggggtggatttccagtcatggtgcagcttaggt
atgaacctggccaggctcatatgagcatgggggggtctattacagaaaccagccccgagcaaaggggttctga
agtccataccaaaaagacagtgatgatcaagaccattgagacccgggatggagaggtgagtggtgtcggaccc
cttgtctgacagcccggtgtctttcaccagctgggtggcttccaaccctggatggggtgggtgggccttgctg
ccttggggtggaacagtttggggtgaggctcttctaagccagcagtggatagactggccttctcctcctgctt
aggttgtcagcgaggctacacagcaacaacatgaagtgctg
3′ HA:
(SEQ ID NO: 27)
gcaagaaattcagtgtcctggccccgtcctcactgcctcctgaagccagcctcttccactctcggatatcaca
cccagccacttttctccactcacaggctctgaccccccctcaccgatcacccctttgtggtcttcatgctgcc
caaccccagggaacccctcagccacctctgcagaccctcccatgagccctggctattggcaggtgtcaaagct
ggctcttaagagagaacccagctcaagtcatcgcccttccccttccacctttgtgacccctggcttaggagag
ggtaccagagagggtgttgggatctgcagggtcaggaccgagtttgtggacatccccagcctgggtcagagac
agaatgaagcctcagcgagctgagatggagagtggggggcctgaaaactgccctcatggcccctctctttccc
atcgcagcccaggatggccttggaaagcgggggctgtaagagggaagcggaaggtgctggatgtgggagcagg
agctacagaaggagagaggatgggtgaggagctggagaggaaggaagagagaggcagagagtgggctcaggtt
ggtgggagggtaccacctcccctgcctgcccctcccaccgcaggggcctggacagaaacaataataaagagac
aagcacaaacctgcatggccctgtcatcttgactgtgtctcagggggagtctggaccgtgtcacacccccatt
gccctgggatgaactgtgtcagtgtcatgctcagcgagattgaacagtaggcctctgggtgctgtagtctggg
gagcgtggggtgctggagaaggggtttctgttaaaaagccacagagccttcggtccagccttctatcccctta
ggctacctatccccttaatatcacctgggcatcgggtaaggtagggatggggacagtcctattctacagactg
gctgtgaaggaagttccctcggtccttttgtccccaaggcactgccagtccctttccattga

### REFERENCES

1. Guo Y, VanDusen NJ, Zhang L, Gu W, Sethi I, Guatimosim S, Ma Q, Jardin BD, Ai Y, Zhang D, Chen B, Guo A, Yuan G-C, Song L-S, Pu WT. Analysis of Cardiac Myocyte Maturation Using CASAAV, a Platform for Rapid Dissection of Cardiac Myocyte Gene Function In Vivo. Circ Res. 2017; 120:1874-1888.
2. Nishiyama J, Mikuni T, Yasuda R. Virus-Mediated Genome Editing via Homology-Directed Repair in Mitotic and Postmitotic Cells in Mammalian Brain. Neuron. 2017; 96:755-768.e5.
3. Yeh CD, Richardson CD, Corn JE. Advances in genome editing through control of DNA repair pathways. Nat Cell Biol. 2019; 21:1468-1478.
4. Ishizu T, Higo S, Masumura Y, Kohama Y, Shiba M, Higo T, Shibamoto M, Nakagawa A, Morimoto S, Takashima S, Hikoso S, Sakata Y. Targeted Genome Replacement via Homology-directed Repair in Non-dividing Cardiomyocytes. Sci Rep. 2017; 7:9363.
5. Kohama Y, Higo S, Masumura Y, Shiba M, Kondo T, Ishizu T, Higo T, Nakamura S, Kameda S, Tabata T, Inoue H, Motooka D, Okuzaki D, Takashima S, Miyagawa S, Sawa Y, Hikoso S, Sakata Y. Adeno-associated virus-mediated gene delivery promotes S-phase entry-independent precise targeted integration in cardiomyocytes. Sci Rep. 2020; 10:15348
6. Juane Lu, Tao Wu, Biao Zhang, Suke Liu, Wenjun Song, Jianjun Qiao & Haihua Ruan, Cell Communication and Signaling volume 19, Article number: 60 (2021)
7. The Cell: A Molecular Approach. 2nd edition.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for integrating an exogenous sequence into a chromosomal sequence of a eukaryotic cell, the method comprising:
a. introducing into the eukaryotic cell:
(i) at least one RNA-guided endonuclease comprising at least one nuclear localization signal or nucleic acid encoding at least one RNA-guided endonuclease comprising at least one nuclear localization signal,
(ii) at least one guide RNA or a DNA encoding at least one guide RNA, and
(iii) at least one donor polynucleotide comprising the exogenous sequence;
b. generating a double-stranded break a target site in the chromosomal sequence, wherein at least one guide RNA guides the at least one RNA-guided endonuclease to the target site; and
c. repairing the double strand break using a DNA repair process,
thereby integrating the exogenous sequence into the chromosomal sequence of the eukaryotic cell,
wherein the efficiency of integrating the exogenous sequence is about 20%, 25%, 30%, 35%, 40%, or 45% higher compared to a reference sample.

2. The method of claim 1, wherein the eukaryotic cell is a cardiomyocyte.

3. The method of claim 1, wherein the insertion site for the exogenous sequence is selected from the group consisting of: Mb, Des, Actc1, Cox6a2, Fabp3, Myh6, Rplp1, Acta1, Myl3, Myl2, Myl7, Pln, and Ttn.

4. The method of claim 3, wherein exogenous sequence is integrated into the 5′ or 3′ of Mb or Des.

5. (canceled)

6. A homology directed repair (HDR) construct comprising a left and right homology arm for a genomic edit to be incorporated at a target locus.

7. The HDR construct of claim 6, wherein the target locus is selected from the group consisting of: Mb, Des, Actc1, Cox6a2, Fabp3, Myh6, Rplp1, Acta1, Myl3, Myl2, Myl7, Pln, and Ttn.

8. The method of claim 6, wherein the genomic edit is incorporated into the 5′ or 3′ of Mb or Des.

9. (canceled)

10. The HDR construct of claim 6, further comprising a positive selection or negative selection marker.

11. The HDR construct of claim 6, further comprising a fluorescent marker for FACS isolation of positive cell pools, wherein the fluorescent marker comprises mScarlet, Blue-TagBFP, Cyan-Cerulean, Green-Tag GFP2, Yellow-YPet, Red-TagRFP, Far Red-mKate2.

12. A homology directed repair (HDR) vector comprising the construct of claim 6.

13. The vector of claim 12, wherein the backbone of the vector enables uniform, one-step assembly for incorporating homology arms.

14. The HDR vector of claim 12, wherein the vector is a transfection delivery vector.

15. The HDR vector of claim 12, wherein the vector is a viral delivery vector wherein optionally the viral delivery vector is a lentivirus vector.

16. (canceled)

17. The HDR vector of claim 15, wherein the viral vector is an AAV vector, wherein optionally the AAV vector is an AAV9 vector.

18. (canceled)

19. An engineered, non-naturally occurring CRISPR-Cas system comprising:
a Cas9 protein which is a Streptococcus pyogenes Cas9 comprising mutation or an ortholog thereof having a corresponding mutation, and the HDR vector of claim 6.

20. An isolated, engineered, non-naturally occurring cell comprising the CRISPR-Cas system of claim 19.

21. (canceled)

22. (canceled)

23. The cell of claim 20, wherein the cell is a cardiomyocyte, wherein optionally the cardiomyocyte is a skeleton muscle cell.

24. (canceled)

25. A method of treating a disease in a subject, comprising administering an effective amount of the HDR construct of the engineered non-naturally occurring CRISPR-Cas system of claim 19 to the subject, thereby treating the subject.

26. The method of claim 25, wherein the subject is a human subject.

27. The method of claim 25, wherein the disease is a cardiomyopathy or a skeletal myopathy.


SEQUENCE LISTING (27 sequences)

SEQ ID NO: 1 - DNA, 20 bp, synthetic construct
  source 1..20: mol_type = other DNA
agtgggctgtgggtcacctg

SEQ ID NO: 2 - DNA, 500 bp, synthetic construct
  source 1..500: mol_type = other DNA
gtcacaggcgtgcattcagccacagtccccaccaagccctctcgcccctgagcatagccctgcccagcagctccttcaacagctggatctccattttcccctgctgggcatctgggaggttgcggggaggtctaggggacttcagatcaccccggtgagtaaaggtggggatggtgaagacacaagaacacgtggacccagaagttaatccaggacagtagcctgggcttagttcccagagagaggtaacttttgggcctgctgacttccctcccacctcaagtcctgctgacagcagtcacagcctggctgggggtattggggtccttcccaggatctcaggcccactctgcgccccccttgccattctgaggatctctgaatcccactactcttgcttgcagatcgaccagatgttcgcagcctttccccctgacgtcaccggcaatcttgattataagaatttggtccacatcattacccacggagaagagaaagac

SEQ ID NO: 3 - DNA, 500 bp, synthetic construct
  source 1..500: mol_type = other DNA
gccctgaaccacaggatcaggtgacccacagcccactctccatcccagggctgtgcgcaaataaacaggaagtcttggctctggctgtggtgaccattggctcatttggctctaagtagccaaagtcacaaagtatttgggttctgtgaaggctgcagggatctcagtttgaacagcaaacaggcaccacctggcagtggtggcactttgtggggggcacactccagccattgggagtcagaggcaggtggatctctgagttctaggctagcctggtctacagagtcagttccaagaagtccacggacttcaggagcatctgcatccaggagtggcagttctgtcactctctccaggtggccttcccttagctctggttgctttctgggcccaggctttgaaggtgggaaaaaagacggatggatctctgggacttccagacttagcccgctttacatagtgggtttcaggctagcctgggctacataatgagatcctga

SEQ ID NO: 4 - DNA, 20 bp, synthetic construct
  source 1..20: mol_type = other DNA
ggtcatcctgaatataaggt

SEQ ID NO: 5 - DNA, 500 bp, synthetic construct
  source 1..500: mol_type = other DNA
cacagatacggaactttaggactgcttctaaatagtgaataaaagcaaatatctgaaacacggtgtgtgtgtggggggggggggaagcaataactaaacctatctacacatttaaaaggtccccagcgtttgaactatccttcatgaatattattctttagtacaaataagcagagaaatgatgaaatacaggaaggattgcaaagcctgattggctacattgcttattgtatagcatctcctacgttgtctactatcggttaatctgtgatggtttttctgtgaatattccaagcctgttgcccacttcattgttggggacttcagaacacactgatggttttgttttctctctgcagattattgctccccctgagcgtaaatactctgtctggattgggggctccatcctggcctctctgtccaccttccagcaaatgtggatcagcaagcaagagtatgatgaggcaggcccatccattgtccaccgcaagtgcttc

SEQ ID NO: 6 - DNA, 500 bp, synthetic construct
  source 1..500: mol_type = other DNA
gatgtctctctcttagcataccttatattcaggatgaccgtattgtgcttcttggaatcctctgagccccctccccatctctcatcagtcattgtacagtttgtttacacaagtgcagtttgtttgtgcttcaaatatttattgctttataaataaaccagaccaggacttgcaacctacaaaagcctgtgttctttcttgtgtgagtgggcctgggatggagaaggtgttcactctgatactccgagcattacaatattagtgcaacacaaagtttacccaatgaattcatgtggtgttgaattgagctaggagttacagaggtacatcttttgggatggagttatgaatatttttgaatcttataagcaaagattagttctgtggtttagttgttaggtttagttctgtataagtaaggaatgaagatagaaaacacaattatctacaaccagttgccatgtttggtactaaagttgctttagcacatatcacatatt

SEQ ID NO: 7 - DNA, 20 bp, synthetic construct
  source 1..20: mol_type = other DNA
caaattggccttctgcacac

SEQ ID NO: 8 - DNA, 500 bp, synthetic construct
  source 1..500: mol_type = other DNA
gggcaggaggtaagtggggccaggcctgactctgatttagtagcctagccgatctgttctctttgcccggcttccccctccttctgtctcctggctcctctctcctgatctaccaggcactgatctttcagacctctaggcccagggatagtttttgcctatgaatctactcacaccccacctttgctcctccagccaacacctggcgcctcctgacctttgtgctggctcttcccggcgtagccctctgctcccttaactgctggatgcacgctggccaccacgagcgcccagagttcatcccgtatcaccacctccgcatccgaaccaaggtacgccagaggatgagcgagtgagagcatgcagggggattgcggggagggactgtggtgggtgacctgtctgccttctctctgcagcccttcgcctggggggacggcaaccacacgcttttccacaatccccacgtcaatcctttgcccaccggttatgagcaccct

SEQ ID NO: 9 - DNA, 500 bp, synthetic construct
  source 1..500: mol_type = other DNA
tgtctcagcagacacgctctgccagcaatcttcaaattggccttctgcacaccagctctgagagcccctgaggttccagtggacagttccaagctcaataaaggtgtggaagttttgtgtcctctggctctttgggaacagcatggtggaaggggctgggcaggctcttgggcagttggtatctgggttccagttatttttttttatataaggaaaatgtgatgtttccacctgcattttcattttattttttaaaaatttatacaagcaatacattgaatattgagcatattactcagtcttgtgttagttcttatccatatgtaaatttatgctgaccccattcctgctcttgcaaaaaaagtggatcagcactgaaatctgacacatgggcaaaccataaaaatctcccagctcaggaggtagggcatcatttctgggaagtttcccaggctagcccagagctgaagagaacttgttcccatgccagtagattcata

SEQ ID NO: 10 - DNA, 20 bp, synthetic construct
  source 1..20: mol_type = other DNA
ccagttggcagaggagcggg

SEQ ID NO: 11 - DNA, 500 bp, synthetic construct
  source 1..500: mol_type = other DNA
gaagggcctagctctggaagggaggtaacaaagaggatcatgttcactaggtagctagagtaaggtgaggccatggctgggctctacagtgccagaatactgcatgttaggggagggaggaaaagttggcagcttagcagtttctcaaggctctgttcttatcacccgtttgtctgccacatacaccaggcacccccttaggcaggtgctgaaatgaacacaaaggaggaacaggaatgtactggatcttgaccagtttacctcctctctcaagggcctatttttcccccaaatctctaaaatgctaattataacatcttaaaagatttgtatcagaaaaaaaagtaaagtgcctggcacacagtaggtgctcaagtgctggtcaggatgagggtggggagcactccctcctctgctctgccccatctgaaacctgtctttctttctagactctcactcatggcagtgtggtgagcactcggacttatgagaaggaggcg

SEQ ID NO: 12 - DNA, 500 bp, synthetic construct
  source 1..500: mol_type = other DNA
cctggctgctccgtcactgacagcccgctcctctgccaactggccacccctcagctcagcaccatgctgcctcatggttttcccctctgacattttgtataaacattcttgggttgggatttttctggagatacggggcatcagcctggacccagttcctactatgtatgtggtttattttttaaaactgtatccaaagggtgctccaaggtcaataaagcagaaccaaggccacccagttgtctgtctttggtcctcctttcctgtgtgtcaggttgaaatgaaggcctataggtcacctgggaagcagcactgtcaaggagccgagtggacaggctcaaggctcagttagggaacagtagcacctatgtaatacccttacactgacctgccaaggctcagagaagctagctgtcattctagcatctatgcaagcccttacactggcctgcccatggcagagcagctggctgtcactgtgtggctatttcacattcatc

SEQ ID NO: 13 - DNA, 20 bp, synthetic construct
  source 1..20: mol_type = other DNA
tctccagcagaccctcgctg

SEQ ID NO: 14 - DNA, 500 bp, synthetic construct
  source 1..500: mol_type = other DNA
agcaggccaacaccaacctgtccaagttccgcaaggtgcagcacgagctggatgaggcggaggagagggcggacatcgccgagtcccaggtcaacaagctgcgggccaagagccgggacattggtgccaaggtgagttcctcccttggaactgctagtcacgcgctcatcaggatgcccctgcaagcatgatgctctgaacagctacgtgtgtatcccatgtgcatttagacatagaataggcatacatacaaccgaagggtcccacagacacctgtaacctgagagtccactctcccacttgaagtgggccccagatctgtgttctaaactacaagcactgatatcatgagacttcaagactgctgaggttcaacccctcccttcccaagggcattttattagagccactgaaatccctagaaattcctccccaggccatcgtacccactgacctcacatctcttactcctttctcagcagaagatgcacgacgaggaa

SEQ ID NO: 15 - DNA, 500 bp, synthetic construct
  source 1..500: mol_type = other DNA
cctctccagcagaccctcgctgtagccaatccacaataaacataaacgttcgactctgcctgcaccctgtccttcctaccaagcgtctctctggggtgggaattttggtggtccaggaggagggtggccacagcagggactgtcagagctgtaagatctgaagcaccccatggtctgtggaagccgagagagaagtgaggtgtcttgcctcaggcagcacagctacagccccagctccacttggtcgttgtgcaagtatagaaagggaaactttccaaacgtgggggaaacaaatccaggtgtgagggatggccaagcctcagatgggaggtggaggcaagaggcaggctcagctagaaggcaagtttaaagccagcctgggtagcgtgagagctcacctcaaagacaagaaaacaacaacccctcaatgagtccaaagaacagaggcactgaaagcgagccaagtcagatccagggggatggaagtttgctgacgagct

SEQ ID NO: 16 - DNA, 20 bp, synthetic construct
  source 1..20: mol_type = other DNA
ctaaactgcttttgttaagt

SEQ ID NO: 17 - DNA, 500 bp, synthetic construct
  source 1..500: mol_type = other DNA
cttagtttgtacttattgatgtggcttttatgtcaggccttcacggtccatggcttgagaatgagttttgttgcctcttaagtttgggcatttgaagcagtgagattcatgcaaagtggatagctacattctatttatacaaacagctttatttctggcaagtggggtcaatcaactggtggcagttgtatattgattccctgtagtcctgaacattgctttggtctcatgggtgaaaagagcttaattgaatcattaaatcttgcagcacaaaactaaaagccttttctatgtctctgttgtaggctctggccaatgtcaacattgggagcctcatctgcaatgtaggggctggtgggcccgctccagcagctggagctgcgcctgctggtggtgctgctccatccactgccgccgccccagctgaggagaagaaagtggaggcaaagaaggaagagtccgaggagtctgaagatgacatgggcttcggtctttttgac

SEQ ID NO: 18 - DNA, 500 bp, synthetic construct
  source 1..500: mol_type = other DNA
actgcttttgttaagttagctaataaagagctgaacctgtaggtggactggtctcacttgtaagccacaaagctcttggagtttcagagatggtagcaggtcccaaaaaaatgtcagggaaggagggtcttgaccaaagccaccatgagttatataaataagacctgggtcaaaagaactagacgatctgggtagagaatgggtgggtctgtctccaggaagccccttcaagcaactgttcagagagcctgtttagtgccagacagacaggctgagagctaactcttactgtcaagaataacaaaataacctctgggtctcccgatgttcatgtgtgaagaacttgacagttgttgaggatctcagaagcatctagaagggccagagaacctaccagtccactggtgtaaaaccagcagcctaaaggattcagtcacgcagcaaggcttggtgtgtaactacaacttcatgtagaaagtttaggacttggggctgacagt

SEQ ID NO: 19 - DNA, 20 bp, synthetic construct
  source 1..20: mol_type = other DNA
cagcttggtgggctggacag

SEQ ID NO: 20 - DNA, 500 bp, synthetic construct
  source 1..500: mol_type = other DNA
ccggctgctgggcctgcatcctgaagtagtgtgggcacacgagaggtgagaaaggggccccagaggggctccctgagtttgagtgaccctccctgactgcatgacttcatgcacacagctatacctgtctttgcctcagtttccccatttacagagcagggatggtggtggtgcctgcttccaggtagtcaggtttaaaggagttggttcatggtaattgcatggaatggcacttggtacatcgtaagtgtgcaaggaacagtcctatagtaaggagaaggtcagtgagtacacacccccttagctcatggcttgctccgtcccctgacctacaacctcttgtccctttcttgcagtttatctcagaaattatcattgaagtcctgaagaagagacattccggggactttggagcagatgctcagggcgccatgagcaaggccctggagctcttccggaatgacattgccgccaagtacaaggagctaggcttccagggc

SEQ ID NO: 21 - DNA, 500 bp, synthetic construct
  source 1..500: mol_type = other DNA
gccatgggctccaactgtccagcccaccaagctgggacccagtgttgtgtagcaagtagcgtgtgcagtgttctaggttagcagagaacagaagaggggagcatagtgtggcatccacccacacccctggggacagggctctgggcagtgttaccctggagcccagaggtgcaaagtggccttcgtcagctctgccgggtcatgctcaggtctcctaagtcccagtccattttcttctggttttgggaaaatctcttttccactgtcacatttgaccccaaatccaagtcactgactagcagaccctgacctttgggcgagatggagggttgcttagagggagtggagggtgaaaacggggcggtgagcatcaagtctcccactgctcagcttcccgttgacccaccttgtctcaataaaatatcctgcgagtcctcaattcttgtctccgtccggtatatttttatcccttccggtaagggaaacatccacaaacgtcg

SEQ ID NO: 22 - DNA, 20 bp, synthetic construct
  source 1..20: mol_type = other DNA
ggcttccagggctgagccat

SEQ ID NO: 23 - DNA, 1005 bp, synthetic construct
  source 1..1005: mol_type = other DNA
tgcaaagatgtcctgtgcctctgggtgcacttgtgtccctcggtgtccccttctgctgtgtcccactctcatctctgctgtgtcacttgcacataagggccccagcccaaaacagcagctgtacccccactcctgagctagcaggggtgtgtccacagtccctgggtctaatctcagcttactatctacctgctacatgacctcagcctggtcacttggtagtctcttcagtacagcagccaaagctgtacgggaccaaactctaagtcaatgacgataagtaagtgttatgatagtttcatcgtgagcctttggattctgggtggtcccgatccccatctaggcaatgtcggtacctgatctcccctggcccctctccccttgttagtacctgttcctcagtcccaggtttagggataaaatcacatgatgggaaaagcctcctcccaagtgtcctgattgacagtcctggtgggctagccgatgctgagcaaggtatcatgggccggctgctgggcctgcatcctgaagtagtgtgggcacacgagaggtgagaaaggggccccagaggggctccctgagtttgagtgaccctccctgactgcatgacttcatgcacacagctatacctgtctttgcctcagtttccccatttacagagcagggatggtggtggtgcctgcttccaggtagtcaggtttaaaggagttggttcatggtaattgcatggaatggcacttggtacatcgtaagtgtgcaaggaacagtcctatagtaaggagaaggtcagtgagtacacacccccttagctcatggcttgctccgtcccctgacctacaacctcttgtccctttcttgcagtttatctcagaaattatcattgaagtcctgaagaagagacattccggggactttggagcagatgctcagggcgccatgagcaaggccctggagctcttccggaatgacattgccgccaagtacaaggagctaggcttcaagagc

SEQ ID NO: 24 - DNA, 952 bp, synthetic construct
  source 1..952: mol_type = other DNA
gccatgagctcccactgtccagcccaccaagctgggacccagtgttgtgtagcaagtagcgtgtgcagtgttctaggttagcagagaacagaagaggggagcatagtgtggcatccacccacacccctggggacagggctctgggcagtgttaccctggagcccagaggtgcaaagtggccttcgtcagctctgccgggtcatgctcaggtctcctaagtcccagtccattttcttctggttttgggaaaatctcttttccactgtcacatttgaccccaaatccaagtcactgactagcagaccctgacctttgggcgagatggagggttgcttagagggagtggagggtgaaaacggggcggtgagcatcaagtctcccactgctcagcttcccgttgacccaccttgtctcaataaaatatcctgcgagtcctcaattcttgtctccgtccggtatatttttatcccttccggtaagggaaacatccacaaacgtcgctggaagaaatgggaaggtgcattctgggatggcgggcagacagaagatgtgagacaggcccctttctacaatcacctctcaatgacaagcacacactgggctaggttcagggacagggaagaaagggccccccacctgggctctgatcaagatggcgggcctgcagacagggtcagctgaccttggcagtctgaggatcagtgccaggtgcccagggcaggagacagggaggagatgtctcgctgtggatccaacagctgaggcaatgtgagaaggtcacccagccagaacgtagcagagctgagagccaaacagtggcctagaagacaactgggtctctgaccccaggacagagcctgtggccaacagtagcagctgctgaccttccggccagggagaggacagacaacagggagatactatccaaacacaggacaggggaagaaaccca

SEQ ID NO: 25 - DNA, 20 bp, synthetic construct
  source 1..20: mol_type = other DNA
gggccaggacactgaattcc

SEQ ID NO: 26 - DNA, 991 bp, synthetic construct
  source 1..991: mol_type = other DNA
ctagttgcttgctaagggactcatgagcgccaaactcctgacaccacggaactaaatgtgacccccactatgaaaactcagatttcttttttcacttatttgtttattcagataccaagtatgtgttctgcatataaaatcacttttgctgcggatcagcagctctgagacagaataagtggactatcatggctgctacttcccagagaggaaactgaggcacaggagggactagctcctgcatcattaagacattaaacagctcatcctggagcctctaggttcctccagcaggtctataggcctgggctttgaggtcctgtcttagatgagagtccacggatataaactcaccattcaggactcttctgaaaatctggaggtggagagagagcaaggcaggataggaaacatgaatactgttcccgggccttgggtcccctgctatcctgtaccaacacacagcacaatcttttggctcttgaagccattcctctagcaagactggtccctctctctataaatcatctgttgggtgctgggttccaggatgaggtctcaaagaagtcagacagttgtcataagaaaggtgaaagtcagctagcacttggagcaatgcagcctggggtggatttccagtcatggtgcagcttaggtatgaacctggccaggctcatatgagcatggggtgggtctattacagaaaccagccccgagcaaaggggttctgaagtccataccaaaaagacagtgatgatcaagaccattgagacccgggatggagaggtgagtggtgtcggaccccttgtctgacagcccggtgtctttcaccagctgggtggcttccaaccctggatggggtgggtgggccttgctgccttggggtggaacagtttggggtgaggctcttctaagccagcagtggatagactggccttctcctcctgcttaggttgtcagcgaggctacacagcaacaacatgaagtgctg

SEQ ID NO: 27 - DNA, 1011 bp, synthetic construct
  source 1..1011: mol_type = other DNA
gcaagaaattcagtgtcctggccccgtcctcactgcctcctgaagccagcctcttccactctcggatatcacacccagccacttttctccactcacaggctctgaccccccctcaccgatcacccctttgtggtcttcatgctgcccaaccccagggaacccctcagccacctctgcagaccctcccatgagccctggctattggcaggtgtcaaagctggctcttaagagagaacccagctcaagtcatcgcccttccccttccacctttgtgacccctggcttaggagagggtaccagagagggtgttgggatctgcagggtcaggaccgagtttgtggacatccccagcctgggtcagagacagaatgaagcctcagcgagctgagatggagagtggggggcctgaaaactgccctcatggcccctctctttcccatcgcagcccaggatggccttggaaagcgggggctgtaagagggaagcggaaggtgctggatgtgggagcaggagctacagaaggagagaggatgggtgaggagctggagaggaaggaagagagaggcagagagtgggctcaggttggtgggagggtaccacctcccctgcctgcccctcccaccgcaggggcctggacagaaacaataataaagagacaagcacaaacctgcatggccctgtcatcttgactgtgtctcagggggagtctggaccgtgtcacacccccattgccctgggatgaactgtgtcagtgtcatgctcagcgagattgaacagtaggcctctgggtgctgtagtctggggagcgtggggtgctggagaaggggtttctgttaaaaagccacagagccttcggtccagccttctatccccttaggctacctatccccttaatatcacctgggcatcgggtaaggtagggatggggacagtcctattctacagactggctgtgaaggaagttccctcggtccttttgtccccaaggcactgccagtccctttccattga